SEQUENCE LISTING (52 sequences)

SEQ ID NO: 1 - DNA, 23 bp, Artificial Sequence
  23 nb 5′UTR-glpF (the pin structure)
cgtggaggtccgtgactttcacg

SEQ ID NO: 2 - DNA, 54 bp, Artificial Sequence
  54nb 5′UTR-glpF
tgcctacaagcatcgtggaggtccgtgactttcacgcatacaacaaacattaac

SEQ ID NO: 3 - DNA, 16 bp, Artificial Sequence
  Rec RBS ( 6UTR)
caaggaggaaacagct

SEQ ID NO: 4 - DNA, 16 bp, Artificial Sequence
  recRBS_v1 (SD1)
caaattcgaaacagct

SEQ ID NO: 5 - DNA, 16 bp, Artificial Sequence
  recRBS_v2 (SD2)
caagcgcaaaacagct

SEQ ID NO: 6 - DNA, 16 bp, Artificial Sequence
  recRBS_v3 (SD3)
caagaacaaaacagct

SEQ ID NO: 7 - DNA, 16 bp, Artificial Sequence
  recRBS_v4 (SD4)
caactaggaaacagct

SEQ ID NO: 8 - DNA, 16 bp, Artificial Sequence
  recRBS_v5 (SD5)
caaccgagaaacagct

SEQ ID NO: 9 - DNA, 16 bp, Artificial Sequence
  recRBS_v6 (SD6)
caagagctaaacagct

SEQ ID NO: 10 - DNA, 16 bp, Artificial Sequence
  recRBS_v7 (SD7)
caagagcaaaacagct

SEQ ID NO: 11 - DNA, 16 bp, Artificial Sequence
  recRBS_v8 (SD8)
caagagaaaaacagct

SEQ ID NO: 12 - DNA, 16 bp, Artificial Sequence
  recRBS_v9 (SD9)
caaaggaaaaacagct

SEQ ID NO: 13 - DNA, 16 bp, Artificial Sequence
  recRBS_v10 (SD10)
caactgagaaacagct

SEQ ID NO: 14 - DNA, 16 bp, Artificial Sequence
  16 nb lacZ RBS
cacacaggaaacagct

SEQ ID NO: 15 - DNA, 16 bp, Artificial Sequence
  16 nb glpF RBS
tcttcaggatccgatt

SEQ ID NO: 16 - DNA, 16 bp, Artificial Sequence
  16 nb glpA RBS
cttcagagggataaca

SEQ ID NO: 17 - DNA, 16 bp, Artificial Sequence
  16 nb glpA RBS
gccacggaggctatca

SEQ ID NO: 18 - DNA, 16 bp, Artificial Sequence
  glpD RBS
agtgaatgagggcagc

SEQ ID NO: 19 - DNA, 16 bp, Artificial Sequence
  16 nb gatY RBS
acgacaggatatgaaa

SEQ ID NO: 20 - DNA, 16 bp, Artificial Sequence
  16 nb mglB RBS
aaaaaccggagatacc

SEQ ID NO: 21 - DNA, 250 bp, Artificial Sequence
  PgatY_org
cggcaacctatgcctgatgcgacgctgaagcgtcttatcatgcctacatagcactgccacgtatgtttacaccgcatccggcataaaaacacgcgcactttgctacggcttccctatcgggaggccgtttttttgcctttcactcctcgaataattttcatattgtcgtttttgtgatcgttatctcgatatttaaaaacaaataatttcattatattttgaaatcgaaaacaaacgacaggatatgaaa

SEQ ID NO: 22 - DNA, 350 bp, Artificial Sequence
  PmglB_org
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagaaaaaccggagatacc

SEQ ID NO: 23 - DNA, 291 bp, Artificial Sequence
  PgatY_54UTR
cggcaacctatgcctgatgcgacgctgaagcgtcttatcatgcctacatagcactgccacgtatgtttacaccgcatccggcataaaaacacgcgcactttgctacggcttccctatcgggaggccgtttttttgcctttcactcctcgaataattttcatattgtcgtttttgtgatcgttatctcgatatttaaaaacaaataatttcattatattttgtgcctacaagcatcgtggaggtccgtgactttcacgcatacaacaaacattaacacgacaggatatgaaa

SEQ ID NO: 24 - DNA, 203 bp, Artificial Sequence
  PmglB_54UTR
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 25 - DNA, 350 bp, Artificial Sequence
  PmglB_16UTR
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 26 - DNA, 203 bp, Artificial Sequence
  PmglB_70UTR
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 27 - DNA, 203 bp, Artificial Sequence
  PmglB_70UTR_SD4
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 28 - DNA, 203 bp, Artificial Sequence
  PmglB_70UTR_SD5
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 29 - DNA, 203 bp, Artificial Sequence
  PmglB_70UTR_SD7
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 30 - DNA, 203 bp, Artificial Sequence
  PmglB_70UTR_SD8
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 31 - DNA, 203 bp, Artificial Sequence
  PmglB_70UTR_SD9
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 32 - DNA, 34 bp, Artificial Sequence
  Oligo for amplification of plasmid backbone (forward)
attaacccuccaggcatcaaataaaacgaaaggc

SEQ ID NO: 33 - DNA, 24 bp, Artificial Sequence
  Oligo for amplification of insert in galK site (forward)
cccagcgagacctgaccgcagaac

SEQ ID NO: 34 - DNA, 24 bp, Artificial Sequence
  Oligo for amplification of insert in galK site (reverse)
ccccagtccatcagcgtgactacc

SEQ ID NO: 35 - DNA, 28 bp, Artificial Sequence
  Oligo for amplification of lacZ (forward)
aaacagcuatgaccatgattacggattc

SEQ ID NO: 36 - DNA, 40 bp, Artificial Sequence
  Oligo for amplification of lacZ (reverse)
agggttaautgcgcgttatttttgacaccagaccaactgg

SEQ ID NO: 37 - DNA, 32 bp, Artificial Sequence
  Oligo for amplification of plasmid backbone (reverse)
atttgcgcaucaccaatcaaattcacgcggcc

SEQ ID NO: 38 - DNA, 35 bp, Artificial Sequence
  Oligo for amplification of PgatY-promoter elements (forward)
atgcgcaaaucggcaacctatgcctgatgcgacgc

SEQ ID NO: 39 - DNA, 37 bp, Artificial Sequence
  Oligo for amplification of PmglB-promoter elements (forward)
atgcgcaaautgcgtcgccattctgtcgcaacacgcc

SEQ ID NO: 40 - DNA, 45 bp, Artificial Sequence
  Oligo for amplification of PmglB_16UTR promoter element (reverse)
agctgttucctccttgcttatgcagggtagtgcttgagataaatg

SEQ ID NO: 41 - DNA, 36 bp, Artificial Sequence
  Oligo for amplification of PmglB_70UTR_SD4 promoter element (reverse)
agctgttucctagttggttaatgtttgttgtatgcg

SEQ ID NO: 42 - DNA, 36 bp, Artificial Sequence
  Oligo for amplification of PmglB_70UTR_SD5 promoter element (reverse)
agctgttuctcggttggttaatgtttgttgtatgcg

SEQ ID NO: 43 - DNA, 36 bp, Artificial Sequence
  Oligo for amplification of PmglB_70UTR_SD7 promoter element (reverse)
agctgttutgctcttggttaatgtttgttgtatgcg

SEQ ID NO: 44 - DNA, 36 bp, Artificial Sequence
  Oligo for amplification of PmglB_70UTR_SD8 promoter element (reverse)
agctgttuttctcttggttaatgtttgttgtatgcg

SEQ ID NO: 45 - DNA, 36 bp, Artificial Sequence
  Oligo for amplification of PmglB_70UTR_SD9 promoter element (reverse)
agctgttuttcctttggttaatgtttgttgtatgcg

SEQ ID NO: 46 - DNA, 95 bp, Artificial Sequence
  Oligo for amplification of PmglB_70UTR promoter element (reverse)
agctgttucctccttggttaatgtttgttgtatgcgtgaaagtcacggacctccacgatgcttgtaggcacggtgcaatcatagctatcacattg

SEQ ID NO: 47 - DNA, 35 bp, Artificial Sequence
  Oligo for amplification of PmglB_org promoter element (reverse)
atggtcauggtatctccggtttttcttatgcaggg

SEQ ID NO: 48 - DNA, 33 bp, Artificial Sequence
  Oligo for amplification of PgatY_org promoter element (reverse)
atggtcautttcatatcctgtcgtttgttttcg

SEQ ID NO: 49 - DNA, 103 bp, Artificial Sequence
  Oligo for amplification of PmglB_54UTR promoter element (reverse)
atggtcauggtatctccggtttttgttaatgtttgttgtatgcgtgaaagtcacggacctccacgatgcttgtaggcacggtgcaatcatagctatcacattg

SEQ ID NO: 50 - DNA, 100 bp, Artificial Sequence
  Oligo for amplification of PgatY_54UTR promoter element (reverse)
atggtcautttcatatcctgtcgtgttaatgtttgttgtatgcgtgaaagtcacggacctccacgatgcttgtaggcacaaaatataatgaaattatttg

SEQ ID NO: 51 - DNA, 22 bp, Artificial Sequence
  CRP__gatY
ttttgtgatcgttatctcgata

SEQ ID NO: 52 - DNA, 22 bp, Artificial Sequence
  CRP__mglB
atctgtgagtgatttcacagta

(19) United States
(12) Patent Application Publication
(10) Pub. No.: US 2022/0267782 A1
(43) Pub. Date: Aug. 25, 2022

(54) NUCLEIC ACID CONSTRUCT COMPRISING 5' UTR STEM-LOOP FOR IN VITRO AND IN VIVO GENE EXPRESSION

(71) Applicant: Glycom A/S, Hørsholm (DK)

(72) Inventor: Margit PEDERSEN, Roskilde (DK)

(21) Appl. No.: 17/596,781

(22) PCT Filed: Jun. 19, 2020
(86) PCT No.: PCT/IB2020/055773

§ 371 Date: Dec. 17, 2021

(30) Foreign Application Priority Data
Jun. 21, 2019 (DK) PA 2019 00756

(51) Int. Cl. C12N 15/70; C12N 15/113

(52) U.S. Cl. CPC C12N 15/70; C12N 15/113; C12N 2310/531

(57) ABSTRACT

The present invention relates to the field of recombinant production of biological molecules in host cells. The invention provides nucleic acid constructs that allow to modify expression of a desired gene using both in vitro and in vivo gene expression systems with optimized stem-loop structures in the 5′ UTR of said genes. The constructs can advantageously be used to produce a variety of biological molecules recombinantly in industrial scales, e.g. human milk oligosaccharides (HMOs)

### CROSS-REFERENCE TO RELATED APPLICATION

This application is a national stage entry pursuant to 35 U.S.C. § 371 of International Application No. PCT/IB2020/055773 filed on Jun. 19, 2020 which claims priority to Denmark Patent Application No. PA 2019 00756 filed on Jun. 21, 2019, the contents of all of which are fully incorporated herein by reference.

### FIELD OF THE INVENTION

The present invention relates to the field of recombinant production of biological molecules in host cells. The invention provides nucleic acid constructs that allow to modify expression of a desired gene using both in vitro and in vivo gene expression systems. The constructs can advantageously be used to produce a variety of biological molecules recombinantly in industrial scales, e.g. human milk oligosaccharides (HMO).

### BACKGROUND OF INVENTION

The commercial importance of recombinant microorganisms to produce biological molecules is increasing. Currently, production of recombinant proteins in bacterial hosts, in particular E. coli, mostly uses plasmid-borne expression systems. Since these systems provide high gene dosage and the available cloning protocols are simple to handle, they have become widely accepted. However, usage of plasmid-based expression systems, especially on a manufacturing scale has a bundle of downsides as well.

The genome-based expression systems seem to have a great potential to ensure stable and selection-marker-free expression of recombinant genes, compared to the plasmid-based expression systems. However, often expression of a recombinant gene on a manufacturing scale is achievable only by increasing the gene dosage in the chromosome to the plasmid level, as a single copy of the gene is often not able to provide a satisfactory expression on a manufacturing scale. Furthermore, the selection of a gene integration site is a challenge, and the regulation of expression is often complex and/or not suitable for industrial production. Consequently, there are not many simple robust and effective genome-based bacterial expression system for industrial production of recombinant polypeptides available at present.

One approach to overcome the problem of insufficient level of production and complex regulation of genome-based bacterial expression of heterologous polypeptides is the use of strong inducible promoters for controlling the transcription of integrated recombinant genes. A number of different inducible promoters have been described and examined as alternatives to commonly used IPTG inducible promoters, like lac, e.g. promoters induced by high temperatures, such as λP_{R} and λP_{L}, tryptophan starvation, such as trp, 1, arabinose, such as araBAD, mannitol, such as mtsE, phosphate starvation such as phoA, nalidixic acid such as recA, osmolarity such as proU, glucose starvation, such as cst-1, etc. However, there is a number of problems associated with used of these inducible promoters, e.g. the induction conditions may be harmful for cells, produced molecules and/or equipment, or they make purification more costly and difficult.

Use of recombinant carbon source regulated promoters is probably the most attractive option for controlling expression of the target genes in industrial settings. The reasons for this is that these promoters are regulated by the availability of a carbon source which allows for recombinant gene expression in a controlled environment which reduces the extend of metabolic stresses on the host cell otherwise introduced by the inducer. However, at present the choice of such promoters is rather limited, and most of the available have been adopted for plasmid-borne expression. Still, the genome of a bacterial cell, e.g. E. coli contains thousands of promoters, and many of them are regulated by changes in the carbon source, allowing carbon availability in the environment to influence the expression pattern of genes under their control. It has been suggested that the global transcription regulator, cAMP-CRP or CRP, which is formed when glucose is limited, regulates a minimum of 378 promoters of a bacterial cell (Shimada T. et al., PloS One 6(6): e20081, (2011)), however, there is no data that would suggest which of these promoters are suitable for driving a genome-based stable controllable high-yield production of recombinant biological molecules in industrial settings. Furthermore, high levels of expression of recombinant genes controlled by these promoters (i.e. production of RNA and/or polypeptides) cannot always be achieved despite efficient and high promoter activity because other regulatory mechanisms on the transcription and/or translation level play an important role in the regulation of gene expression as well.

Recently, a new recombinant bacterial expression system comprising nucleic acid constructs where a promoter element is fused with a synthetic DNA sequence that comprises an artificial ribosomal binding site has been described (WO2019/123324). The described expression system allows modulating the level of expression of a gene both in vivo and in vitro. The system utilizes recombinant nucleic acid constructs comprising a g/p promoter element operably linked to a synthetic DNA sequence comprising a fragment derived form the genomic 5′UTR sequence located upstream of the glpF gene of E. coli and a particular recombinant DNA sequence comprising a ribosomal binding site.

### SUMMARY OF INVENTION

A first aspect of the invention relates to an isolated nucleic acid consisting of SEQ ID NO: 1, or a variant thereof, or a complementary nucleic acid sequence thereof, wherein said variant is a nucleic acid sequence that has at least 80%, preferably, more than 80% sequence identity with SEQ ID NO:1.

A second aspect of the invention relates to a contiguous synthetic nucleic acid comprising a DNA sequence (i) and a promoter element operably linked to said DNA sequence (i),
wherein
(a) the DNA sequence (i) has the length of at least 23 nucleobases and comprises SEQ ID NO:1, or a variant thereof; wherein said variant has at least 80% sequence identity with SEQ ID NO:1; and
(b) the promoter element is an isolated DNA sequence that comprises a single binding site for cyclic AMP receptor protein (CRP), wherein said site is centred at position around −41 upstream the transcription start point.

In different embodiments the construct may further comprise a DNA sequence (ii), wherein said DNA sequence (ii) is operably linked to the DNA sequence (i) and positioned downstream the DNA sequence (i). The DNA sequence (ii) in some embodiments may be a non-coding DNA sequence and in other embodiments it may be a coding DNA sequence. In some embodiments, the DNA construct may comprise a further coding DNA sequence.

A third aspect of the invention relates to a nucleic acid construct comprising a contiguous synthetic nucleic acid comprising two DNA sequences (i) and (ii), wherein the sequences are operably linked and the DNA sequence (ii) is located downstream the DNA sequence (i), and, wherein
(a) the DNA sequence (i) has the length of at least 23 nucleobases and comprises SEQ ID NO:1, or a variant thereof;
(b) the DNA sequence (ii) does not comprise any of the sequences of SEQ ID NOs: 3-18;

In one embodiment, a construct of the third aspect further comprises an operably linked promoter element. In one embodiment, the promoter element comprises a DNA sequence that comprises a single binding site for the Cyclic AMP Receptor Protein (CRP), which site is centred at position around −41 upstream the transcription start point.

In some embodiments, a construct of the second and/or third aspect may comprise a coding DNA sequence that encodes a functional polypeptide, such as an enzyme, transport protein, antigen, regulatory protein, or a small non-coding RNA molecule, such as a regulatory microRNA (miRNA) or small interfering RNA (siRNA).

In a fourth aspect, the invention relates to a vector comprising an isolated nucleic acid sequence of the first aspect or a nucleic acid construct of the second or third aspect.

In a fifth aspect, the invention relates to an expression cassette comprising an isolated nucleic acid sequence of the first aspect or a nucleic acid construct of the second and/or third aspect

In a sixth aspect, the invention relates to an expression system comprising an isolated nucleic acid sequence of the first aspect, a nucleic acid construct of the second and/or third aspect, a vector of the fourth aspect, and/or an expression cassette of the fifth aspect.

In a seventh aspect, the invention relates to a recombinant cell, preferably a bacterial recombinant cell, comprising a synthetic nucleic acid, a nucleic acid construct, vector and/or expression cassette of the first, second, third, fourth, fifth aspect, correspondingly.

In a eighth aspect, the invention relates to a method of recombinant production of one or more biological molecules, e.g. a protein, nucleic acid, oligosaccharide, such as a human milk oligosaccharide (HMO), etc, using a synthetic nucleic acid and/or construct and/or vector and/or expression system, and/or recombinant cell of the first, second, third, fourth fifth, sixth, seventh aspect of the invention.

These and further aspects of the invention are described in detail below.

### BRIEF DESCRIPTION OF DRAWINGS

FIG. 1 is a schematic presentation of an embodiment of a nucleic acid construct of the invention.

FIG. 2 presents the expression levels of a reporter gene (lacZ) from nucleic acid constructs comprising synthetic promoter elements that originate from the operons, gatYZABCDR, and mglBAC, i.e. PgatY_org and PmglB_org, fused to a promoter-less lacZ reporter gene and integrated into the chromosomal DNA in a single copy (open bars). The gene expression control elements were modified by replacing the original 5′UTR DNA sequence located between the transcriptional start site and the 16^{th} nucleotide upstream the translational start codon with SEQ ID NO: 2. The expression levels of lacZ from the different expression cassettes were measured. The data shows the level of activity of the expressed β-galactosidase in host cells. The activity was measured in Miller Units (U/OD/ml/min).

FIG. 3 presents the expression levels of a reporter gene (lacZ) from nucleic acid constructs comprising eight different gene expression control elements. The synthetic promoter element originates from the operon mglBAC, i.e. PmglB_org. The data shows the level of activity of β-galactosidase expressed in host cells from eight different constructs comprising eight variants of the RBS sequence. The activity was measured in Miller Units (U/OD/ml/min). The eight constructs comprise a gene expression control element having the sequences as the following: SEQ ID:22 (PmglB_org); SEQ ID NO: 25 (PmglB_16UTR); SEQ ID NO: 29 (PmglB_70UTR_SD7); SEQ ID NO: 28 (PmglB_70UTR_SD5); SEQ ID NO:26 (PmglB_70UTR); SEQ ID NO: 31 (PmglB_70UTR_SD9); SEQ ID NO: 30 (PmglB_70UTR_SD8); SEQ ID NO: 27 (PmglB_70UTR_SD4).

FIG. 4 presents the predicted secondary structure of the transcript of SEQ ID NO: 2 using the RNAfold WebServer (http://rna.tbi.univie.ac.at/cgi-bin/RNAWebSuite/RNAfold.cgi). The stem-loop structure formed by SEQ ID NO:1 is outlined.

### DETAILED DESCRIPTION OF INVENTION

The present invention relates to synthetic nucleic acids, DNA constructs and expression systems comprising thereof useful for modulating of gene expression and recombinant production of biological molecules in vivo and in vitro. According to the invention, recombinant nucleic acids, constructs and bacterial expression systems described herein are capable of modulating of expression of genes both in vitro and in vivo, such as increasing or decreasing expression of a genomic or recombinant DNA sequence of interest. By “expression of a gene” is meant production of the gene products, i.e. RNA or polypeptide molecule(s), in a recombinant cell or cell-free expression system comprising a nucleic acid or construct of the invention. In particular, the invention relates to recombinant nucleic acid sequences, such as nucleic acid constructs, comprising an isolated nucleic acid consisting of SEQ ID NO: 1, or a variant thereof, wherein said variant is a nucleic acid sequence that has at least 80%, preferably, more than 80% sequence identity with SEQ ID NO:1. It was found that a transcript of the DNA sequence of SEQ ID NO: 1 is capable of forming a stem-loop (pin) structure that is associated with an increased stability of an RNA molecule that comprises this structure. Nucleic acid constructs comprising this DNA sequence of the invention can significantly increase efficiency of expression of genes operably linked to the constructs in recombinant cells by increasing the lifetime of genes transcripts (i.e. mRNA) and, consequently, the number of cycles of translation of the transcripts. Advantageously, constructs of the invention may comprise carbon source regulated promoters that have a single binding site for CRP at position −41, which facilitates regulation of expression of a gene linked or included in the construct.

Embodiments of nucleic acids and constructs of the invention are described below.

Unless otherwise specified, all technical and scientific terms used herein have the same meaning as commonly understood by one of ordinary skill in the art to which this invention belongs. Singleton et al. (1994) Dictionary of Microbiology and Molecular Biology, second edition, John Wiley and Sons (New York) provides one of skill with a general dictionary of many of the terms used in this invention. Although any methods and materials similar or equivalent to those described herein can be used in the practice or testing of the present invention, the preferred methods and materials are described. Most of the nomenclature and general laboratory procedures required in this application can be found in Sambrook et al., Molecular Cloning: A Laboratory Manual, Vol. 1-3, Cold Spring Harbor Laboratory, Cold Spring Harbor, N.Y. (2012); Wilson K. and Walker J., Principles and Techniques of Biochemistry and Molecular Biology (2010), Cambridge University Press; or in Maniatise et al., Molecular Cloning A laboratory Manual, Cold Spring Harbor Laboratory (2012); or in Ausubel et al., Current protocols in molecular biology, John Wiley and Sohns (2010). The manuals are hereinafter referred to as “Sambrook et al.”, “Wilson & Walker”, “Maniatise et al”, “Ausubel et al”, correspondingly.

If not otherwise specified, the terms defined throughout specification relate to all aspects and embodiments of the invention. All embodiments described in specification and working examples relate to all and any aspects of the invention.

The term “nucleic acid construct” means an artificially constructed segment of nucleic acid, in particular a DNA segment, which is intended to be used for expression of recombinant genes or non-coding regulatory RNA molecules, like miRMA or siRNA molecules, in vivo or in vitro, or for modification of expression of genes or DNA sequences encoding regulatory RNA molecules that are naturally comprised in the genomic DNA of a target organism in which the nucleic acid construct is to be ‘transplanted’. Accordingly, a construct of the invention, in different embodiments, may or may not comprise a coding DNA sequence, i.e. a DNA sequence encoding a polypeptide, or a DNA sequence encoding a regulatory RNA molecule, e.g. a siRNA or miRNA molecule.

In some preferred embodiments, a nucleic acid construct comprises a contiguous DNA sequence that includes two distinct fragments that are operably linked together: a promoter DNA sequence, a synthetic DNA sequence comprising SEQ ID NO: 1. In different embodiments the synthetic DNA sequence may comprise one DNA sequence, a DNA sequence (i), wherein the DNA sequence (i) comprises SEQ ID NO: 1, or it may comprise two linked DNA sequences: DNA sequence (i) and DNA sequence (ii) that does not comprise SEQ ID NO: 1. In other preferred embodiments, nucleic acid constructs may comprise a synthetic DNA sequence that comprises DNA sequence (i) and, optionally, DNA sequence (ii), that is not linked to a promoter DNA sequence (i.e. a promoter-less construct). Yet, in some other preferred embodiments, a construct may comprise a synthetic DNA sequence comprising only a DNA sequence (i) that is operably linked to a promoter DNA sequence. These embodiments are useful for regulation of expression target genomic sequences, e.g. a gene, in the genome of a host microorganism. Such constructs can be incorporated into the genome of the host upstream the transcription site point of any gene or other genomic sequences. In other preferred embodiments, a construct may further comprise both, a promoter DNA sequence and a synthetic DNA sequence of the invention, and further comprises one or more coding DNA sequences that are operably liked to the DNA sequences controlling the gene expression of the construct (i.e. the promoter sequence and the synthetic DNA comprising SEQ ID NO:1). Different embodiments of these constructs are described below throughout the specification and illustrated by non-limiting working examples.

As used herein, the term “nucleic acid” includes RNA, DNA and cDNA molecules. It is understood that, as a result of the degeneracy of the genetic code, a multitude of nucleotide sequences encoding a given protein may be produced. A nucleic sequence that encodes a functional biological molecule, e.g. peptide, polypeptide or nucleic acid, e.g. an sRNA, is termed “coding DNA sequence”. A nucleic acid that does not encodes a functional biological molecule is termed “non-coding DNA sequence. The term nucleic acid is used interchangeably with the term “polynucleotide”. The term “oligonucleotide” means a short nucleic acid molecule., e.g. a primer. The term “primer”, means an oligonucleotide, whether occurring naturally in a purified restriction digest or produced synthetically, which is capable of acting as a point of initiation of synthesis when placed under conditions in which synthesis of a primer extension product which is complementary to a nucleic acid strand is induced (i.e. in the presence of nucleotides and an inducing agent such as DNA polymerase and at a suitable temperature and pH). The primer is preferably single stranded for maximum efficiency in amplification but may alternatively be double stranded. If double stranded, the primer is first treated to separate its strands before being used to prepare extension products. Preferably, the primer is a deoxyribonucleotide. The primer must be sufficiently long to prime the synthesis of extension products in the presence of the inducing agent. The exact lengths of the primers will depend on many factors, including temperature, source of primer and the use of the method.

The term “synthetic DNA sequence” means a manmade DNA sequence, i.e. an artificially made DNA sequence. In one preferred embodiment a synthetic DNA sequence of the invention is a contiguous sequence of nucleotides making up a DNA molecule that comprises a DNA sequence (i) and, optionally, a DNA sequence (ii), wherein the two DNA sequences are linked so that the DNA sequence (i) is located upstream the DNA sequence (ii). In another preferred embodiment, a synthetic contiguous DNA sequence of the invention is included in a nucleic acid construct, wherein said synthetic DNA sequence is operably linked to least one promoter element DNA sequence downstream the transcription start. In embodiments when a contiguous DNA sequence of the construct comprises two DNA sequences: a DNA sequence (i) and a DNA sequence (ii), the DNA (i) and (ii) sequences are linked so that the DNA sequence (i) is located upstream the DNA sequence (ii), and a promoter DNA is operably linked to DNA sequence (i) upstream the transcription start. The term “synthetic DNA sequence” is interchangeably used herein with the term “recombinant/artificial DNA sequence”.

The DNA sequence (i) and DNA sequence (ii) in different embodiments can both/either be isolated fragments of a genomic DNA, i.e. deriving from a genomic DNA, e.g. the genomic DNA of Escherichia coli (E coih), and/or artificial DNA sequences (i.e. not deriving from a genomic DNA sequence). The term “isolated DNA sequence” means that the DNA sequence is not an integrated fragment of the genomic DNA, but an artificial/recombinant DNA fragment. Accordingly, the term “isolated DNA sequence” is interchangeably used herein with the term “artificial/recombinant DNA sequence”. In some embodiments of the invention, an isolated DNA sequence may be identical or homologous to a genomic DNA sequence, in other embodiments it may have a nucleotide sequence that has little or no homology to a genomic DNA sequence. The term “homologous” means that a recombinant/isolated DNA fragment has a certain percent of homology (i.e. sequence identity), such as around 65-70%, preferably at least 80%, such 81% to 89%, such as around 90% to around 99%, with a nucleotide sequence which is an integral part of a genomic DNA sequence. The invention also includes recombinant DNA sequences that have the indicated percent of homology to different isolated/recombinant DNA sequences included in nucleic acid constructs of the invention, e.g. a promoter sequence, DNA sequence (i) or DNA sequence (ii). These DNA sequences are referred herein as “variants” of the reference DNA sequence included in the construct of the invention. Preferably, a variant of a reference sequence of a construct of the invention is an artificial nucleic acid sequence that has around 70-99% sequence identity to that particular reference sequence. The scope of term “variant” also includes nucleotide sequences complementary to the DNA sequences described herein, mRNA sequences and synthetic oligonucleotide sequences, e.g. PCR primers. In general, the percentage of identity of the compared nucleic acid sequences indicates the portion of the sequences that has the identical nucleotide composition. In one preferred embodiment, a variant is a reference sequence of a construct of the invention has around 70-99% identity of the nucleotide sequence and the same or a similar function, e.g. it is or can serve as a ribosomal binding site (RBS), or as a binding site for a regulatory protein or enzyme, etc. As mentioned, the scope of the invention also includes nucleic acid sequences that are complementary to DNA sequences of the invention and nucleic acid sequences that are complementary to variants thereof, e.g. RNA sequences. According to the invention, RNA sequences that are complementary to the DNA sequences of variants of reference DNA sequences retain the same structural and functional characteristics as the RNA sequences complementary to reference DNA sequences, e.g. a stem-loop structure. The percentage of sequence identity/homology for the purposes of the invention can be determined by using any method well-known in the art e.g. BLAST.

In one preferred embodiment, the DNA sequence (i) is an isolated DNA fragment of the genomic 5′-untranslated leading DNA sequence (5′UTR DNA) which has at least 80%, preferably more than 80% sequence identity, such as 90-100% sequence identity to a fragment of the genomic 5′UTR DNA of the glpF gene of Escherichia coli (E. coli). Preferably, the fragment comprises a sequence of at least 23 nucleobases, e.g. 23-54 nucleobases, downstream the transcription start (starting from the +2 nucleotide) of the glpF gene, or it is a variant of said sequence of at least 23 nucleotides. Preferably, a 23-nucleobase DNA sequence (i) consists of or comprises SEQ ID NO:1, or a variant thereof. In one preferred embodiment, the DNA sequence (i) consists of SEQ ID NO: 2, or a fragment or variant of SEQ ID NO: 2, wherein said fragment or variant has a length of more that 23 nucleobases and comprises SEQ ID NO: 1, or a variant of SEQ ID NO: 1. Preferably, both variants of SEQ ID NO: 1 or SEQ ID: 2 has at least 80% homology with the reference sequence.

According to the invention the DNA sequence (ii) may be any DNA sequence comprising at least 6 contiguous nucleobases. In one preferred embodiment, a DNA sequence (ii) is a non-coding DNA sequence and comprises a ribosomal binding site that, preferably, has the length of at least 6 nucleobases. By the term “ribosome binding site” (RBS) is meant a nucleotide sequence comprising about 4-16 nucleobases, preferably 6-16 nucleobases, that functions by positioning the ribosome on the mRNA molecule for translation of an encoded polypeptide. In one preferred embodiment the DNA sequence (ii) comprising an RBS is an isolated DNA fragment that has a length of 16 nucleobases. In one preferred embodiment the DNA sequence (ii) comprising an RBS is identical or homologous to a genomic RBS-containing DNA sequence, e.g. to a sequence identified in SEQ ID NOs: 14-20; in another embodiment the RBS-containing DNA sequence (ii) may be an artificial DNA sequence. Non-limiting embodiments of such DNA sequence (ii) are sequences identified in SEQ ID NOs: 3-13. In some preferred embodiments, an RBS of the DNA sequence (ii) does not comprise any of the sequences of SEQ ID NOS: 3-18. In some preferred embodiments, the DNA sequence (ii) comprising a RBS comprises SEQ ID NO: 20 or SEQ ID NO: 19, preferably, the RBS has the sequence of SEQ ID NO: 20 or SEQ ID NO: 19. In some preferred embodiments, the RBS DNA sequence included in constructs of the invention is a sequence selected from any of SEQ ID NO: 4-13. The invention also contemplates a synthetic DNA comprising a DNA (ii) sequence that is not identical to or is a variant of any of the sequences identified in SEQ ID NOs: 3-20.

In another preferred embodiment, a DNA sequence (ii) of the invention is a coding DNA sequence that encodes a functional RNA molecule, such as regulatory RNA molecule e.g. a mall interfering RNA (siRNA), or microRNA (miRNA) molecule. Small interfering RNA (siRNA), sometimes known as short interfering RNA or silencing RNA, is a class of double-stranded RNA non-coding RNA molecules, 20-25 base pairs in length, similar to miRNA, and operating within the RNA interference (RNAi) pathway. It interferes with the expression of specific genes with complementary nucleotide sequences by degrading mRNA after transcription, preventing translation. A microRNA (abbreviated miRNA) is a small non-coding RNA molecule (containing about 22 nucleotides) found in plants, animals and some viruses, that functions in RNA silencing and post-transcriptional regulation of gene expression, miRNAs function via base-pairing with complementary sequences within mRNA molecules. As a result, these mRNA molecules are silenced, by one or more of the following processes: (1) Cleavage of the mRNA strand into two pieces, (2) Destabilization of the mRNA through shortening of its poly(A) tail, and (3) Less efficient translation of the mRNA into proteins by ribosomes. miRNAs resemble the siRNAs, except miRNAs derive from regions of RNA transcripts that fold back on themselves to form short hairpins, whereas siRNAs derive from longer regions of double-stranded RNA.

As mentioned, a nucleic acid construct of the invention in some preferred embodiments comprises a promoter DNA sequence that is operably linked to a synthetic DNA sequence comprising a DNA sequence (i) and, optionally, a DNA sequence (ii) described above.

The term “promoter” or “promoter region” or “promoter element” means a nucleic acid sequence that is recognized and bound by a DNA dependent RNA polymerase during initiation of transcription. The promoter, together with other transcriptional and translational regulatory nucleic acid sequences (also termed “control sequences”) is necessary to express a given gene or group of genes (an operon) to produce the gene-encoded molecules. The “transcription start site” means the first nucleotide to be transcribed and it is designated +1. Nucleotides downstream of the start site are numbered +2, +3, +4 etc., and nucleotides in the 5′ opposite (upstream) direction are numbered −1, −2, −3 etc. A promoter of the invention is an isolated DNA sequence. The promoter DNA of the invention is preferably derived from or homologous to a genomic DNA sequence comprised in the promoter region of a gene. According to the invention any promoter DNA sequence that is able to bind to a DNA dependent RNA polymerase and initiate transcription is suitable for practicing the invention.

As mentioned, a promoter DNA sequence of the invention may derive from the genomic promoter region of any gene, preferably, a gene included in the genomic DNA of E. coli. In a preferred embodiment, a promoter DNA sequence of the invention derives from the promoter region of a gene, which expression is regulated by a carbon source. “Carbon source” refers in general to a carbohydrate molecule, which can be taken up and metabolized by a bacterial cell. The activity of a promoter can be controlled by the presence or absence of a carbon source molecule in the medium, e.g. glycerol, glucose, arabinose, etc. In one preferred embodiment, activity of a promoter of a construct of the invention is a carbon source regulatable. In one preferred embodiment, the DNA sequence of a carbon source regulatable promoter of the invention comprises a single binding site for CRP, wherein said the single CRP-binding site is centred at position around −41. The terms “around”, “about” and “approximately” in general mean a 1-10% deviation of the indicated value, or a minor deviation that does not influence a relevant feature. In the present context, the term “around” means the positions at −39, −39.5, −40, −40.5, −41.5, −42, −42.5 or −43, e.g. the position −40, 5 or −41, 5, in the promoter DNA sequence. Preferably, said CRP-binding site has a length of at least 15 nucleobases and comprises the consensus DNA sequence 5′-TGTGA-N6-TCA(T)C-3′, wherein N6 is a sequence of 6 (any) nucleobases. In one preferred embodiment, the CRP binding site of a promoter has the sequence of SEQ ID NO: 51. In another preferred embodiment, the single CRP binding site has the sequence of SEQ ID NO: 52.

As mentioned, in some embodiments the nucleotide sequence of a promoter DNA of the construct may be identical, or has a certain percent of identity, such as around 65-70%, preferably at least 80% identity, preferably from around 90% to around 99% of identity, to the nucleotide sequence of a fragment of the genomic DNA sequence, preferably, a bacterial genomic DNA sequence, e.g. E. coli genomic DNA, that is regarded as the promoter region of a single gene or an operon. One non-limiting example of such promoter DNA sequence could be a promoter DNA sequence controlling expression of genes of the gatYZABCDR operon of E. coli, in particular, a full-length or fragment of the gatY promoter DNA sequence (abbreviated herein as PgatY); or a DNA sequence of the promoter controlling expression of genes of the mglBAC operon of E. coli, in particular a full-length or fragment of the mglB promoter element (abbreviated herein as PmglB). The E. coli genome is referred herein to the complete genomic DNA sequence of E coli K-12 MG1655 (GenBank ID: U00096.3). Preferred, but not limited embodiments of the promoter DNA sequences of the invention are SEQ ID NO: 21 and SEQ ID NO: 22. Other suitable examples genomic promoter DNA sequences that can be isolated and included in the constructs of the invention could be found in Shimada T. et al., PloS One 6(6): e20081, (2011). In some embodiments, a promoter DNA can be an artificial DNA sequence, i.e. a DNA sequence that is not derived from a genomic promoter sequence.

A promoter DNA sequence of the constructs may comprise various structural features/elements, such as regulatory regions capable of affecting (facilitating or inhibiting) the binding of RNA polymerase in the cell and initiating transcription of the downstream (the 3′-direction) coding sequence, such as e.g. binding sites for transcriptional activators proteins or transcriptional repressors. The regulatory region of a promoter of the invention comprises particular protein binding domains (consensus sequences) responsible for the binding of RNA polymerase such as the −35 box and the −10 box (Pribnow box). All mentioned regulatory sequences of promoter DNA of the construct may have certain percent of identity to the corresponding genomic sequences of the selected promoter, i.e. the invention contemplates the original (native/wild type) DNA sequences or variants thereof.

A promoter sequence of the invention preferably comprises at least 50 nucleotides, more preferably at least 60 nucleotides, such as from around 65 to around 100, from around 75 to around 115 nucleotides, from around 85 to around 125, e.g. 90 to 115, 110-120, 120-130, 130-140, 140-150, or over 150 nucleotides, such as 155-165, 165-175, 175-185, 185-195, 195-205, 205-215, 215-225, 225-235, 235-245, 245-255, 255-265, 250-350. In some embodiment the promoter sequence may be up to 500-1000 nucleotide long. In some embodiments, the selected promoter sequence may also be shorter than 50 nucleotides. In one preferred embodiment, the length of a promoter DNA sequence is at least 50 nucleobases and comprises a single CRP binding site centred at position around −41. In some embodiments, where the DNA sequence (ii) does not comprise any of the sequences identified in SEQ ID NOs: 3-18, the promoter DNA length may be longer or shorter than the sequence of 50 nucleobases and it may comprise several binding sites for CRP or not comprise any CRP binding sites. The length of a promoter DNA sequence of a construct of the invention is not a general limiting factor. Any promoter DNA sequence that is capable of binding to an RNA polymerase and initiate ex-situ or in-situ transcription of a gene may be suitable for the purposes of the invention. In one preferred embodiment, the promoter DNA of the construct has the sequence identified as SEQ ID NO: 21, or has a sequence of a variant of SEQ ID NO:21; in another preferred embodiment, the promoter DNA of the construct has the sequence identified as SEQ ID NO: 22, or has a sequence of a variant of SEQ ID NO:22.

Some embodiments of the invention may relate to non-regulatable promoters, i.e. activity of the promoter does not require initiation, so-called constitutive promoters.

Nucleic acid constructs of the invention may further contain a recombinant coding DNA sequence, which is operably linked to other sequences of the construct. “Operably linked” mean a configuration in which a control sequence (i.e. a promoter sequence) and a. a synthetic DNA comprising a DNA sequence (i) and, optionally, a DNA sequence (ii) are placed appropriately in relation to each other and to a coding DNA sequence, if a coding DNA is included in the construct, i.e. all sequences are placed in the order that the promoter and the synthetic DNA sequence(s) direct the transcription of the coding sequence and translation of the mRNA encoded by the coding DNA. In embodiments where the constructs comprise a coding DNA sequence, preferably, the coding DNA encodes at least one protein or and RNA molecule that has an activity that is directly or indirectly involved in the production of one or more HMOs in the host cell (i.e. the activity of the molecules is essential or beneficial for the production of one or more HMOs). Non-limiting examples of such activities may be an enzymatic, regulatory, chaperone activity. DNA constructs of the invention in some embodiments may comprise more than one coding DNA sequence, which may encode different biological molecules. Preferably, the constructs (containing one or more coding DNA sequences) comprise a single copy of a promoter DNA sequence and a single copy of the synthetic DNA sequence that is operably linked to the promoter. The DNA constructs of the invention may be inserted into a plasmid DNA/vector, transplanted into the target/host cell and expressed as plasmid- and/or chromosome-borne. The DNA constructs may be linear or circular. A linear or circular DNA construct integrated into the host bacterial genome or expression plasmid is interchangeably termed herein as “expression cassette”, “expression cartridge” or “cartridge”. In one embodiment, the expression cassette is a linear DNA construct comprising three DMA sequences: a promoter DNA sequence, a synthetic DNA sequence (as described above) downstream the promoter, and a coding DNA sequence encoding a biological molecule of interest. The construct may also comprise further nucleotide sequences, e. g. a transcriptional terminator sequence, and two terminally flanking regions, which are homologous to a genomic region and enable homologous recombination, and/or other sequences. The cartridge can be made by methods well-known known in the art, e.g. using standard methods described in Wilson & Walker. The use of a linear expression cartridge may provide the advantage that the genomic integration site can be freely chosen by the respective design of the flanking homologous regions of the cartridge. Thereby, integration of the linear expression cartridge allows for greater variability with regard to the genomic region. Linear cartridges are included in preferred embodiments of the invention.

According to the invention, the coding DNA sequence is an isolated DNA sequence that has approximately 70-100% sequence identity to a fragment of genomic DNA that comprise a gene encoding a biological molecule, e.g. protein or RNA. The coding DNA of the construct may be homologous or heterologous to the promoter DNA sequence. “Heterologous” in the present context means that expression of the corresponding genomic coding DNA sequence is naturally controlled by another promoter than the promoter of the construct. Accordingly, “homologous” in the present context means that the corresponding genomic sequences of the promoter DNA sequence and the coding DNA sequence are naturally linked in the genome of species of origin.

In a preferred embodiment, a coding nucleic acid sequence of a construct of the invention is heterologous with respect to the promoter. With respect to the host cell, in which the coding DNA is to be expressed, said DNA may be either heterologous (i.e. derived from another biological species or genus) or homologous (i.e. derived from the host cell). For example, in one embodiment, a coding DNA sequence of the construct may encode a biological molecule, e.g. a protein that is foreign to the host, i.e. the nucleic acid sequence of the coding DNA is heterologous to the host species as it is originating from a donor species which is different from the host organism, or the nucleic acid sequence of the coding DNA contains modification that results in expression of a polypeptide that is not identical to a polypeptide expressed from the corresponding non-modified DNA sequence of the host, i.e. an artificially modified coding DNA sequence originally derived from the host is regarded in the present context as heterologous. In case the host is a particular prokaryotic species, the heterologous nucleic acid sequence may originate from a different genus of family, a different order or class, a different phylum (division), or a different domain (empire) of organisms. The heterologous nucleic acid sequence originating from a donor different from the host can be modified, before it is introduced into the host cell, by mutations, insertions, deletions or substitutions of single nucleic acids or a part of the heterologous nucleic acid sequence as long as such modified sequences exhibit the same function (functionally equivalent) as a reference sequence. A heterologous nucleic acid sequence, as referred herein, encompasses as well nucleic sequences originating from a different domain (empire) of organisms such as from eukaryotes (of eukaryotic origin), such as e.g. enzymes involved in synthesis or degradation of human milk oligosaccharides (HMOs). Still, in other embodiments of the invention, the coding nucleic acid may be homologous with respect to the host cell. The term “homologous nucleic acid sequence” (synonymously used herein as “nucleic acid sequence native to a host” or “nucleic acid sequence derived from the host”) in this context means that the nucleic acid sequence originates (or derives) from the same organism, or same genus of family, or same order or class, the same phylum (division), or same domain (empire) of organisms as the host organism. In one embodiment, the coding DNA of the construct described herein may encode an enzyme or a sugar transporter protein which are normally expressed by the host bacterial cell that naturally comprises in its genome genes encoding said enzyme or sugar transporter protein.

Generally, any coding DNA is contemplated by the invention as any coding DNA can be included in a construct of the invention and transcribed from a promoter included in the construct. In some preferred embodiments the coding DNA encodes a protein, e.g. an enzyme, transport protein, regulatory protein, chaperone, etc. The term “protein” is interchangeably termed herein as “polypeptide”. In other preferred embodiments, the coding DNA might encode a regulatory (non-coding) RNA molecule (ncRNA), e.g. such as functionally important types of RNAs as transfer RNAs (tRNAs) and ribosomal RNAs (rRNAs), as well as small RNAs such as microRNAs, siRNAs, and the long ncRNAs. Preferably, the coding DNA might encode a regulatory (non-coding) RNA molecule which is a small RNA such as a microRNA, a siRNAs. In these embodiments, the construct preferably comprises a promoter element that operably linked to a synthetic DNA sequence that comprises a DNA sequence (i) and do not comprise a DNA sequence (ii). The synthetic DNA (i) is directly linked to a coding DNA encoding a regulatory (non-coding) RNA molecule (ncRNA).

In a preferred embodiment, at least one coding DNA of the construct of the invention encodes a protein or an RNA related to the synthesis, degradation or transport of human milk oligosaccharides, precursors or derivatives thereof. “At least one” means that the construct in different embodiments may comprise more than one coding DNA sequence, e.g. two coding sequences, such as a first and a second coding sequence; three coding sequences, such as a first, a second and a third coding sequence etc. Preferably, multiple coding DNA sequence are in these embodiments are expressed as tandem, and the transcription is controlled by a single copy of the promoter DNA of the construct. The first, second, third, etc. coding DNA sequences may in different embodiments encode for different enzymes or other proteins that function is essential or beneficial for the HMO production by a host cell, e.g. enzymes, transporter proteins, regulatory proteins, chaperones, etc. By “essential” in the present context is meant that the protein is involved in the HMO synthesis directly, e.g. it is an enzyme that assists the process of making an HMO from the HMO precursor, e.g. an enzyme with glucosyltransferase activity. By “beneficial” in the present context is meant that the protein is not involved in the HMO synthesis directly, but it assists a process that is beneficial for the HMO production by a host cell, e.g. it a protein that assists transport (into or out of the host cell) of an HMO or an HMO precursor. Some not-limiting embodiments of proteins, which are regarded herein essential for the production of one or more HMOs by a host cell can be found in the prior art, e.g. in WO20191233324 (see Tables 2 and 3—incorporated herein by reference).

The term “human milk oligosaccharide” or “HMO” in the present context means a complex carbohydrate found in human breast milk (for ref see Urashima et al.: Milk Oligosaccharides. Nova Science Publisher (2011); or Chen, Adv. Carbohydr. Chem. Biochem. 72, 113 (2015)). The HMOs have a core structure comprising a lactose unit at the reducing end that can be elongated by one or more β-N-acetyl-lactosaminyl and/or one or more β-lacto-N-biosyl units, and this core structure can be substituted by an α L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-1), sialyl-lacto-N-neohexaose II (SLNH-II) and disialyl-lacto-N-tetraose (DSLNT). In the context of the present invention lactose is regarded as an HMO species.

The term “HMO precursor” in the present context refers to a compound being involved in the biosynthetic pathway of one or more HMOs according to the invention, which are produced and naturally present in the host cell or imported into the cell from the extracellular medium. Some non-limiting examples of HMO precursors are listed below:

| Precursor: | Product: |
|---|---|
| UDP-GlcNAc | LNT, LNnT, LNFP-I, LNFP-II, LNFP-III, LNFP-V, LNFP-VI, LNDFH- |
| | I, LNDFH-II, pLNH, F-pLNH I, 3′SL, 6′SL, pLNnH, (F)LSTa, |
| | (F)LSTb, (F)LSTc, (F)LSTd |
| UDP-Gal | LNT, LNnT, LNFP-I, LNFP-II, LNFP-III, LNFP-V, LNFP-VI, LNDFH- |
| | I, LNDFH-II, pLNH, F-pLNH I, pLNnH, LSTa, LSTb, LSTc, LSTd |
| GDP-fucose | LNT, LNnT, LNFP-I, LNFP-II, LNFP-III, LNFP-V, LNFP-VI, LNDFH- |
| | 1, LNDFH-II, F-pLNH I, 2′FL, 3′FL, DFL, FLSTa, FLSTb, FLSTc, |
| | FLSTd |

The term “HMO transporter” means a biological molecule, e.g. protein, that facilitates transport/export an HMO synthesized by the host cell through a cellular membrane, e.g. into the cell medium, or transport/import of an HMO from the cell medium into the cell cytosol.

The term “HMO derivative” means a molecule that is derived from an HMO molecule or comprise an HMO moiety, e.g. a ganglioside molecule, an artificial carbohydrate/protein structure comprising an HMO moiety.

An expression cassette of the invention may be utilized for recombinant production of one or more HMOs either as genome integrated or plasmid-borne, or, in some embodiments, the host cell may comprise both a genome integrated and a plasmid-borne expression cassette, wherein at least one or both of the expression cassettes comprise one or more genes that are essential and/or beneficial for the production of one or more HMOs and wherein the expression of at least one of said genes is under the control of a promoter of the invention, e.g. PmglB or PgatY. Preferably, a genome integrated cassette comprises at least one (or a first set of) coding DNA sequences, and the plasmid-borne cassette comprises at least one second coding DNA (or a second set of coding DNA sequences), wherein the at least one first and/or at least one second coding DNA sequences are operably linked to a promoter of the invention. In some preferred embodiments, at least one of the expression cassettes is expressed under control of PmglB or PgatY, e.g. a coding sequence of the genome integrated cassette is operably linked to a promoter of the invention, e.g. PmglB or PgatY, and the plasmid-borne coding sequence is operably linked to another promoter, e.g. lac promoter or another promoter. In some embodiments, both genome-integrated, and plasmid-borne cassettes may be expressed under the control of the same or a different promoter of the invention, e.g. the promoter of a genome integrated cassette is PmglB and the plasmid-borne promoter is PgatY. In other embodiments, all expression cassettes comprised in the host cell may comprise the same promoter. In one preferred embodiment, the host cell comprises at least one copy of a genome-integrated expression cassette of the invention comprising PmglB or PgatY. Preferably, the host cell genome comprises a single or low number of copies of the genome integrated expression cassette, such as two or three copies. Still, in some embodiments, the host may comprise multiple copies of an expression plasmid, wherein each plasmid comprises a single copy of an expression cassette of the invention. In some embodiments, the host cell may comprise several different nucleic acid constructs of the invention, both/either genome integrated and/or plasmid-borne. Each of the several different nucleic acid constructs may be integrated in the genome of the host cell or into a plasmid in a single or multiple copy. In some embodiments, it is preferred that the constructs are integrated in a single copy or a low copy number.

According to the invention, a single copy of the expression cassette of invention comprised in a host cell either as genome integrated or plasmid-borne can provide an amount of a biological molecule encoded by a coding DNA sequence (preferably, under control of PmglB or PgatY), that is sufficient to secure high production levels of one or more HMOs by the host cell. Surprisingly, a single genome-integrated copy of an expression cassette of the invention can provide the production levels of an HMO that are comparable to or higher (such as 2-10-fold higher) than the production levels achieved using a high number plasmid-borne expression (100-500 copies) of the same cassette. In some embodiments, it may be advantageous to express two or more genes related to the HMO production in in the host cell. The HMO-related genes may be included in one construct and expressed as tandem from a single (or multiple) copy as genome- or plasmid-borne; or the genes may be included in different constructs of the invention and one gene is expressed from the genome integrated cassette and another gene from the plasmid-borne. In other embodiments, other mode of expression, composition, or number of copies of the expression cassettes may be contemplated. Preferably, at least one gene included in later expression cassettes encodes for a protein with an enzymatic activity that is essential for the synthesis of an HMO in the host cell. Non-limiting embodiments of genes that may advantageously be expressed under the control of PmglB or PgatY are described in WO2019123321 (incorporated herein by reference).

According with the above, one aspect of the invention relates to a recombinant cell comprising a nucleic acid construct of the invention as any of the described above. The recombinant cell is interchangeably termed herein as “host cell”. Preferably, the host cell is a bacterial cell. The terms “host bacteria species”, “host bacterial cell” are used interchangeably to designate a bacterial cell that has been transformed to contain a DNA construct of the invention and is capable to express the heterologous polypeptide encoded by corresponding heterologous coding DNA sequence of the construct. The terms “transformation”, “transformed”, and “transplanted” are synonymous and denote a process wherein an extracellular nucleic acid, like a vector comprising a construct of the invention, with or without accompanying material, enters a host cell. Transformation of appropriate host cells with, for example, an expression vector can be accomplished by well-known methods such as, electroporation, conjugation, or by chemical methods such as Calcium phosphate-mediated transformation and by natural transformation systems, described, for example, in Maniatis et al., or in Ausubel et al.

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be Erwinia herbicola (Pantoea agglomerans), Citrobacter freundii, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be modified using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species for the invention described herein. Also included as part of this invention are strains, modified as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa). Bacteria comprising the characteristics described herein are cultured in the presence of lactose, and an HMO produced by the cell is retrieved, either from the bacterium itself or from a culture supernatant of the bacterium. The HMO is purified using a suitable procedure available in the art (e.g. such as described in WO2015188834, WO2017182965 or WO2017152918) In a preferred embodiment, the host cell is E. coli. However, as mentioned, a variety of host cells can be used for the purposes of the invention.

One requirement to the host cell is that it contains a functional DNA-dependent RNA polymerase that can bind to the promoter and initiate transcription of the DNA of the construct. The RNA polymerase may be endogenous (native), homologous (recombinant) or foreign/heterologous (recombinant) to the host cell.

The construct of the invention transformed into a selected bacterial host can be expressed as a genome integrated expression cassette or cloned into a suitable expression vector and expressed as plasmid-borne. In different embodiments it may be preferred to utilize the genome-based expression system, in other embodiments, the plasmid-born expression may be preferred. However, it is an advantage to use the construct of the invention in the genome-based expression system, as, surprisingly, a single copy of the construct integrated into and expressed from the genome can provide a high and stable level of expression of the integrated gene product. In additional advantage is that the genomic expression is sustainable for long periods of time. For the purposes of the invention there can be used standard methods for integration of the constructs of invention into the host cell genome or into expression plasmids which are e.g. described in Sambrook et al., Wilson & Walker, “Maniatise et al, and Ausubel et al.

For the genome-based expression, there is a requirement to a host cell—the cell should be able to carry out homologous recombination (which is relevant for integration of the expression cartridge into the genome). Therefore, the host cell preferably carries the function of the recombination protein RecA. However, since RecA may cause undesirable recombination events during cultivation, the host cell preferably has a genomic mutation in its genomic recA site (rendering it dysfunctional), but has instead the RecA function provided by a recA sequence present on a helper plasmid, which can be removed (cured) after recombination by utilizing the helper plasmid's temperature-sensitive replicon (Datsenko K. A. and Wanner B. L., (2000) Proc Natl Acad Sci USA. 97(12):6640-5). In view of recombination, in addition to RecA, the host cell preferably contains, DNA sequences encoding recombination proteins (e.g. Exo, Beta and Gam). In this case, a host cell may be selected that already has this feature, or a host cell is generated de novo by genetic engineering to insert these sequences.

With regard to the integration locus, the expression system used in the invention allows for a wide variability. In principle, any locus with known sequence may be chosen, with the proviso that the function of the sequence is either dispensable or, if essential, can be complemented (as e.g. in the case of an auxotrophy). Many integration loci suitable for the purposes of the invention are described in the prior art (see e.g. Francia VM & Lobo JMG (1996), J. Bacteriol v178 p. 894-898: Juhas M et al (2014) doi.org/10.1371/ournal.pone.0111451; Juhas M & Aijoka F W (2015) Microbal Biothechnol v. 8:617-748; Sabi A et al (2013) Microbial Cell Factories 12:60).

Integration of the gene of interest into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2(2):137-49.); methods for genomic integration of nucleic acid sequences in which recombination is mediated by the Red recombinase function of the phage A or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. 2005), 12(3):349-56.; Vetcher et al., Appl Environ Microbiol. (2005); 71(4):1829-35).

The DNA construct may also be inserted sited-specific. In view of site-specific gene insertion, another requirement to the host cell is that it contains at least one genomic region (either a coding or any functional or non-functional region or a region with unknown function) that is known by its sequence and that can be disrupted or otherwise manipulated to allow insertion of a heterologous sequence, without being detrimental to the cell.

In certain embodiments, the host cell carries, in its genome, a marker gene in view of selection.

When choosing the integration locus, it needs to be considered that the mutation frequency of DNA caused by the so-called “adaptive evolution” varies across the genome of E. coli and that the metabolic load triggered by chromosomally encoded recombinant gene expression may cause an enhanced mutation frequency at the integration site. In order to obtain an expression host cell that is robust and stable, a highly conserved genomic region that results in a lowered mutation frequency is preferably selected as integration site. Such highly conserved regions of the E. coli genome are for instance the genes encoding components of the ribosome or genes involved in peptidoglycan biosynthesis, and those regions may be preferably selected for integration of the expression cartridge. The exact integration locus is thereby selected in such a way that functional genes are neither destroyed nor impaired, and the integration site should rather be located in non-functional regions.

The genomic region with known sequence that can be chosen for integration of the cartridge may be selected from the coding region of a non-essential gene or a part thereof; from a dispensable functional region (i.e. promoter, transposon, etc.), from genes the deletion of which may have advantageous effects in view of production of a specific protein of interest, e.g. certain proteases, outer membrane proteins, potential contaminants of the product, genes encoding proteins of metabolism (e.g. relevant for the metabolism of a sugar molecule that is undesirable or dispensable for a given host strain and/or fermentation process) or stress signalling pathways, e.g. those occurring in stringent response, a translational control mechanism of prokaryotes that represses tRNA and rRNA synthesis during amino acid starvation. Alternatively, the site of integration may be a marker gene which allows selection for disappearance of said marker phenotype after integration. Alternatively, the site useful to select for integration is a function which, when deleted, provides an auxotrophy, i.e. the inability of an organism to synthesize a particular organic compound required for its growth. In this case, the integration site may be an enzyme involved in biosynthesis or metabolic pathways, the deletion of such enzyme resulting in an auxotrophic strain. Positive clones, i.e. those carrying the expression cassette, may be selected for auxotrophy for the substrate or precursor molecules of said enzymes. Alternatively, the site of integration may be an auxotrophic marker (a non-functional, i.e. defective gene) which is replaced/complemented by the corresponding prototrophic marker (i.e. a sequence that complements or replaces the defective sequence) present on the expression cassette, thus allowing for prototrophic selection.

In one aspect, the region is a non-essential gene. According to one aspect, this may be a gene that is per se non-essential for the cell. Non-essential bacterial genes are known from the literature, e.g. from the PEC (Profiling the E. coli Chromosome) database http://www.shigen.nig.ac.jp/ecoli/pec/genes.jsp) or from the so-called “Keio collection” (Baba et al., Molecular Systems Biology (2006) 2, 2006.0008). One example for a non-essential gene is RecA. Integrating the expression cassette at this site provides the genomic mutation described above in the context with the requirements on the host cells.

Suitable integration sites, e.g. sites that are easily accessible and/or are expected to yield higher expression rates, can be determined in preliminary screens. Such screens can be performed by generating a series of single mutant deletions according to the Keio collection (Baba et al., 2006) whereby the integration cartridge features, as variable elements, various recombination sequences that have been pre-selected in view of specific integration sites, and, as constant elements, the basic sequences for integration and selection, including, as a surrogate “gene of interest”, a DNA sequence encoding an easily detectable protein under the control of an inducible promoter, e.g. the Green Fluorescent Protein. The expression level of the thus created single knockout mutants can be easily quantified by fluorescence measurement. Based on the results of this procedure, a customized expression level of a desired target protein can be achieved by variation of the integration site and/or number of integrated cartridges.

In the embodiments in which the host cell contains DNA sequences encoding recombination proteins (e.g. Exo, Beta and Gam—either as a feature of the starting cell or obtained by genetic engineering-integration can occur at the genomic site where these recombination protein sequences are located. By integration of the expression cartridge, the sequences coding for the recombination proteins are destroyed or removed and consequently need not, as in the case of plasmid-encoded helper proteins, be removed in a separate step.

Positive clones, i.e. clones that carry the expression cassette, can be selected e.g. by means of a marker gene, or loss or gain of gene function.

In some embodiments, host cells are used that already contain a marker gene integrated in their genome, e.g. an antibiotic resistance gene or a gene encoding a fluorescent protein, e.g. GFP. In this case, the expression cartridge which does not contain a selection marker, is integrated at the locus of the chromosomal marker gene, and positive clones are selected for loss/disappearance of the respective phenotype, e.g. they are selected for antibiotic sensitivity or disappearance of fluorescence, which can be directly visualized on the cultivation plates. These embodiments have the advantage that the marker is either interrupted or completely replaced by the expression cassette, and thus no functional marker sequence is present after integration and does not need to be removed, if undesirable, as in the case of antibiotic resistance genes.

Alternatively, the marker gene is part of the expression cartridge. In the case that the marker used for selection is a gene conferring antibiotic resistance (e.g. for kanamycin or chloramphenicol), positive clones are selected for antibiotic resistance (i.e. growth in the presence of the respective antibiotic). The marker gene (irrespective of whether it is present on the host cell's genome or has been introduced by means of the expression cartridge) can be eliminated upon integration of the cassette.

In certain embodiments, the expression cell may be engineered to carry a defective selectable marker gene, e.g. an antibiotic resistance gene like chloramphenicol or kanamycin, a fluorescent marker or a gene involved in a metabolic pathway of a sugar or an amino acid. In this case, the cartridge with the gene of interest carries the missing part of the marker gene, and by integration the marker gene restores its functionality. By way of example, the cartridge carries the missing part of the marker gene at one of its ends and is integrated directly adjacent to the defective marker gene integrated in the genome, such that the fusion of the two fragments renders the marker gene complete and allows its functional expression. In the case of an antibiotic resistance gene, the cells carrying the expression cassette are resistant against the specific antibiotic, in the case of a fluorescent marker cells can be visualized by fluorescence, and in the case of a metabolic pathway gene, cells obtain the ability to metabolize the respective component. The advantage of this embodiment is that only a short proportion of the marker gene of the cartridge needs to be synthesized, enabling shorter or smaller insertion cartridges compared to prior art.

In certain embodiments, selection of positive clones (i.e. clones that carry the expression cassette) may be carried out by correction (i.e. complementation) of an auxotrophy of the host cell. In such embodiments, a host cell is used that has a mutation that has been chosen to allow selection of positive transformant colonies in an easy way, e.g. a strain that has a deletion or mutation that renders it unable to synthesize a compound that is essential for its growth (such mutation being termed as “auxotrophic marker”). For example, a bacterial mutant in which a gene of the proline synthesis pathway is inactivated, is a proline auxotroph. Such a strain is unable to synthesize proline and will therefore only be able to grow if proline can be taken up from the environment, as opposed to a proline prototroph which can grow in the absence of proline.

Any host cell having an auxotrophic marker may be used. Preferably, mutations in genes required for amino acid synthesis are used as auxotrophic markers, for instance mutations in genes relevant for the synthesis of proline, leucine or threonine, or for co-factors like thiamine. According to the invention, the auxotrophy of host cells is corrected by integration of the missing/defective gene as a component of the expression cartridge into the genome along with integration of the gene of interest. The thus obtained prototrophic cells can be easily selected by growing them on a so-called “minimal medium” (prototrophic selection), which does not contain the compound for which the original host cell is auxotroph, thus allowing only positive clones to grow.

Prototrophic selection is independent of the integration locus. The integration locus for prototrophic selection may be any gene in the genome or at the locus carrying the auxotrophic marker. The particular advantage of prototrophic selection is that no antibiotic resistance marker nor any other marker that is foreign to the host remains in the genome after successful integration. Consequently, there is no need for removal of said marker genes, providing a fast and simple cloning and selection procedure. Another advantage is that restoring the gene function is beneficial to the cell and provides a higher stability of the system.

Alternatively, the marker gene that is inserted into the genome together with the expression cartridge, may be a metabolic gene that allows a particular selection mode. Such a metabolic gene may enable the cell to grow on particular (unusual) sugar or other carbon sources, and selection of positive clones can be achieved by growing cells on said sugar as the only carbon source.

As described above, during long term cultivation of bacteria, adaptive evolution may cause an enhanced mutation frequency at the integration site during expression of the chromosomally encoded recombinant protein. The use of an auxotrophic knockout mutant strain in combination with an expression cartridge complementing the lacking function of the mutant strain (thereby generating a prototroph strain from an auxotroph mutant) has the additional advantage that the restored gene provides benefits to the cell by which the cell gains a competitive advantage such that cells in which adaptive evolution has occurred are repressed. Thereby, a means of negative selection for mutated clones is provided.

In some embodiments (in the case that the protein of interest allows for detection on a single-cell or single-colony basis, e.g. by FACS analysis or immunologically (ELISA)), no marker gene is required, since positive clones can be determined by direct detection of the protein of interest.

The integration methods for obtaining the expression host cell are not limited to integration of one gene of interest at one site in the genome; they allow for variability with regard to both the integration site and the expression cassettes. By way of example, more than one gene of interest may be inserted, i.e. two or more identical or different sequences under the control of identical or different promoters can be integrated into one or more different loci on the genome. By way of example, it allows expression of two different proteins that form a heterodimeric complex. Heterodimeric proteins consist of two individually expressed protein subunits. One example of such protein is an antibody molecule, e.g. the heavy and the light chain of a monoclonal antibody or an antibody fragment; other examples of heterodimeric proteins are CapZ, Ras human DNA helicase II, etc. These two sequences encoding the monomers may be present on one expression cartridge which is inserted into one integration locus. Alternatively, these two sequences may also be present on two different expression cartridges, which are inserted independently from each other at two different integration loci. In any case, the promoters and the induction modes may be either the same or different.

Although the invention allows and can advantageously be practiced for plasmid-free production of biological molecules of interest encoded by the gene of the construct of the invention, it does not exclude that in the expression system of the invention comprises a plasmid that carries sequences to be expressed other than the gene of interest, e.g. the helper proteins and/or the recombination proteins described above. Naturally, care should be taken that in such embodiments the advantages of the invention should not be overruled by the presence of the plasmid, i.e. preferably, such plasmid should be present at a low copy number and should not exert a metabolic burden onto the cell.

The expression system useful in the method of the invention may be designed such that it is essentially or completely free of phage functions.

Summarizing the above embodiments, genome-based expression of the expression cassette of the invention provides the following major advantages:

With respect to the construction procedure of the expression host, the advantages are (i) a simple method for synthesis and amplification of the linear insertion cartridge, (ii) a high degree of flexibility (i.e. no limitation) with respect to the integration locus, (iii) a high degree of flexibility with respect to selection marker and selection principle, (iv) the option of subsequent removal of the selection marker, (v) the discrete and defined number of inserted expression cartridges (usually one or two).

Integration of one or more recombinant genes into the genome results in a discrete and pre-defined number of genes of interest per cell. In the embodiment of the invention that inserts one copy of the gene, this number is usually one (except in the case that a cell contains more than one genomes, as it occurs transiently during cell division), as compared to plasmid-based expression which is accompanied by copy numbers up to several hundred. In the expression system used in the method of the present invention, by relieving the host metabolism from plasmid replication, an increased fraction of the cell's synthesis capacity is utilized for recombinant protein production. A strong expression element of the construct, e.g. PmglB or PgatY, can be applied without adverse effects on host metabolism by reduction of the gene dosage.

As mentioned above, plasmid-based expression systems have the drawback that, during cell division, cells may lose the plasmid and thus the gene of interest. Such loss of plasmid depends on several external factors and increases with the number of cell divisions (generations). This means that plasmid-based fermentations are limited with regard to the number of generations (in conventional fermentations, this number is approximately between 20 and 50). In contrast, the genome-based expression system used in the method of the invention ensures a stable, pre-defined gene dosage for a practically infinite number of generations and thus theoretically infinite cultivation time under controlled conditions (without the disadvantage of the occurrence of cells that do not produce the protein of interest and with the only limitation of potentially occurring natural mutations as they may occur in any gene).

In the case of chemically-inducible promoters, the invention provides the particular advantage that the amount of inducer molecule, when e.g. added in a continuous mode, is directly proportional to the gene dosage per cell, either constant over the entire cultivation, or changing over cultivation time at pre-defined values. Thereby control of the recombinant expression rate can be achieved, which is of major interest to adjust the gene expression rate.

Since the genome-based expression system allows exact control of protein expression, it is particularly advantageous in combination with expression targeting pathways that depend or rely on well-controlled expression.

As described above, the invention allows to design simplified processes, improved process predictability and high reproducibility from fermentation to fermentation. The process of the invention, employing the expression system described above, may be conducted in the fed-batch or in the semi-continuous or continuous mode, whereby the advantages of the genome-encoded expression system are optimally exploited. There are no limitations with respect to process parameters such as growth rate, temperature and culture medium components, except as defined by the host cell's requirements and as pre-defined by the selected promoter.

Another advantage relates to the choice of the inducer molecule: Most of the available systems for high-level expression of recombinant genes in E. coli are lac-based promoter-operator systems inducible by IPTG. The expression system used in the invention allows a carbon-limited cultivation, with continuous or pulse supply of the carbon-source, e.g. lactose, and enables a tight expression rate control with a wide range of unexpansive carbon-source inducers, such as glycerol, fucose, lactose, glucose.

Importantly, the expression system used in the invention has the advantage of providing a high yield of recombinantly produced biological molecules, both regarding the molecule concentration per volume culture medium (i.e. the titre) and regarding the molecule content in the obtained biomass. This feature makes the expression system used in the invention superior compared to prior art expression systems.

Furthermore, the invention offers the advantage that selection of the expression host cell and/or the optimal design of the expression cartridge, can be easily achieved in preliminary screening tests. By way of example, in such preliminary screens a series of linear expression cartridges that vary with respect to at least one element that has an impact on expression properties of the protein of interest (expression level or qualitative features like biological activity), i.e. control elements (e.g. promoter and/or polymerase binding site) and/or sequence of the gene of interest (i.e. different codon usage variants) and/or targeting sequences for recombination and/or any other elements on the cartridge, like secretion leaders, is constructed. The cartridge variants are integrated into the genome of a pre-selected host cell and the resulting expression host variants are cultivated, including induction of protein expression, under controlled conditions. By comparing protein expression, the host cell variant showing the most favourable results in view of an industrial manufacturing process is selected. In a variation of this pre-screening approach, instead of determining the optimal expression cartridge, the optimal bacterial strain may be identified by integrating identical cartridges into a panel of different host cells. Since the integration strategy has the advantage of allowing integration of a discrete number of gene copies (e.g. only one) into the genome, pre-screening of various parameters may be done without interference by plasmid replication or changes in plasmid copy number.

According to the invention, the term “cultivating” (or “cultivation”, also termed “fermentation”) relates to the propagation of bacterial expression cells in a controlled bioreactor according to methods known in the industry.

Manufacturing of recombinant proteins is typically accomplished by performing cultivation in larger volumes. The term “manufacturing” and “manufacturing scale” in the meaning of the invention defines a fermentation with a minimum volume of 5 L culture broth. Usually, a “manufacturing scale” process is defined by being capable of processing large volumes of a preparation containing the recombinant protein of interest and yielding amounts of the protein of interest that meet, e.g. in the case of a therapeutic protein, the demands for clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). The behaviour of an expression system in a lab scale method does not allow to predict the behaviour of that system in the complex environment of a bioreactor.

The expression systems of the invention may be advantageously used for recombinant production on a manufacturing scale (with respect to both the volume and the technical system) in combination with a cultivation mode that is based on feeding of nutrients, in particular a fed-batch process or a continuous or semi-continuous process.

In certain embodiments, the method of the invention is a fed-batch process.

Whereas a batch process is a cultivation mode in which all the nutrients necessary for cultivation of the cells are contained in the initial culture medium, without additional supply of further nutrients during fermentation, in a fed-batch process, after a batch phase, a feeding phase takes place in which one or more nutrients are supplied to the culture by feeding. The purpose of nutrient feeding is to increase the amount of biomass (so-called “High-cell-density-cultivation process” or “HCDC”) in order to increase the amount of recombinant protein as well. Although in most cultivation processes the mode of feeding is critical and important, the present invention is not restricted with regard to a certain mode of feeding.

Feeding of nutrients may be done in a continuous or discontinuous mode according to methods known in the art. The feeding mode may be pre-defined (i.e. the feed is added independently from actual process parameters), e.g. linear constant, linear increasing, step-wise increasing or following a mathematical function, e.g. exponential feeding.

In a preferred embodiment, the method of the invention is a fed-batch process, wherein the feeding mode is predefined according to an exponential function. By applying an exponential feeding mode, the specific growth rate p of the cell population can be pre-defined at a constant level and optimized with respect to maximum recombinant protein expression. Control of the feeding rate is based on a desired specific growth rate p. When a defined medium, as described below, is used, growth can be exactly predicted and pre-defined by the calculation of a biomass aliquot to be formed based on the substrate unit provided.

In another preferred embodiment, an exponential feeding mode may be followed, in the final stages of cultivation, by linear constant feeding.

In another embodiment of the fed-batch process, linear constant feeding is applied. Linear constant feeding is characterized by the feeding rate (volume of feed medium per time unit) that is constant (i.e. unchanged) throughout certain cultivation phases.

In another embodiment of the fed-batch process, linear increasing feeding is applied. Linear increasing feeding is characterized by a feeding rate of feed medium following a linear function. Feeding according to a linear increasing function is characterized by a defined increase of feeding rate per a defined time increment.

In another embodiment of the fed-batch process of the invention, a feedback control algorithm is applied for feeding (as opposed to a pre-defined feeding mode). In a feedback-controlled fed-batch process, the feeding rate depends on the actual level of a certain cultivation parameter. Cultivation parameters suitable for feedback-controlled feeding are for instance biomass (and chemical or physical parameters derived thereof), dissolved oxygen, respiratory coefficient, pH, or temperature. Another example for a feedback-controlled feeding mode is based on the actual glucose concentration in the bioreactor

In another embodiment, bacterial cells carrying a genome-based expression cassette according to the present invention are cultivated in continuous mode. A continuous fermentation process is characterized by a defined, constant and continuous rate of feeding of fresh culture medium into the bioreactor, whereby culture broth is at the same time removed from the bioreactor at the same defined, constant and continuous removal rate. By keeping culture medium, feeding rate and removal rate at the same constant level, the cultivation parameters and conditions in the bioreactor remain constant (so-called “steady state”). The specific growth rate μ can be pre-defined and is exclusively a result of the feeding rate and the culture medium volume in the bioreactor. Since cells having one or more genome-based expression cassettes are genetically very stable (as opposed to structurally and segregationally instable plasmid-based expression systems, or expression systems which genome-inserted cassette relies on genomic amplification), the number of generations (cell doublings) of cells according to the invention is theoretically unlimited, as well as, consequently, cultivation time. The advantage of cultivating a genetically stable genome-based expression system in a continuous mode is that a higher total amount of recombinant protein per time period can be obtained, as compared to genetically unstable prior art systems. In addition, due to the theoretically unlimited time of cultivation, continuous cultivation of cells according to the invention may lead to a higher total protein amount per time period even compared to fed-batch cultivation processes.

Another preferred embodiment refers to semi-continuous cultivation of cells. A semi-continuous cultivation process in the meaning of the invention is a process which is operated in its first phase as a fed-batch process (i.e. a batch phase followed by a feeding phase). After a certain volume or biomass has been obtained (i.e. usually when the upper limit of fermenter volume is obtained), a significant part of cell broth containing the recombinant protein of interest is removed from the bioreactor. Subsequently, feeding is initiated again until the biomass or volume of culture broth has again reached a certain value. This method (draining of culture broth and re-filling by feeding) can be proceeded at least once, and theoretically indefinite times.

With regard to the type of the culture medium used in the fermentation process, there are no limitations. The culture medium may be semi-defined, i.e. containing complex media compounds (e.g. yeast extract, soy peptone, casamino acids, etc.), or it may be chemically defined, without any complex compounds.

Preferably, a “defined medium” is used. “Defined” media (also termed “minimal” or “synthetic” media) are exclusively composed of chemically defined substances, i.e. carbon sources such as glucose or glycerol, salts, vitamins, and, in view of a possible strain auxotrophy, specific amino acids or other substances such as thiamine. Most preferably, glucose is used as a carbon source. Usually, the carbon source of the feed medium serves as the growth-limiting component which controls the specific growth rate.

In the methods of the invention, significantly higher yields are obtained, because growth of bacteria and a high, but physiologically tolerable recombinant gene expression rate can be maintained during the whole production process.

Recombinant bacteria and methods for producing HMOs are well known (see e.g. Priem B et al, (2002) Glycobiology; 12(4):235-40; Drouillard S et al, (2006)Angew. Chem. Int. Ed. 45:1778-1780; Fierfort N & Samain E (2008) J Biotechnol 134:261-265; Drouillard S. et al. (2010) Carbohydrate Research 345 1394-1399; Gebus C et al (2012) Carbohydrate Research 363 83-90; WO2019123324).

To produce HMOs, the HMO-producing bacteria as described herein are cultivated according to the procedures known in the art in the presence of a suitable carbon source, e.g. glucose, glycerol, lactose, etc., and the produced HMO is harvested from the cultivation media and the microbial biomass formed during the cultivation process. Thereafter, the HMOs are purified according to the procedures known in the art, e.g. such as described in WO2015188834, WO2017182965 or WO2017152918, and the purified HMOs are used as nutraceuticals, pharmaceuticals, or for any other purpose, e.g. for research.

Other features and advantages of the invention will be apparent from the description of working examples below, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and therefore not limiting the scope of the invention.

### SELECTED EMBODIMENTS OF THE INVENTION

The below is some selected, but not-limiting, embodiments of the invention.

In one embodiment, the invention relates to an isolated nucleic acid sequence identified in SEQ ID NO: 1. In another embodiment, the invention relates to a variant of SEQ ID NO: 1, wherein said variant has at least 80% sequence identity with SEQ ID NO:1.

Preferably, an isolated nucleic acid sequence identified in SEQ ID NO: 1 comprised in a nucleic acid construct.

In one embodiment, the construct comprises a promoter DNA sequence that is operably linked to a contiguous synthetic DNA sequence (i),
wherein
a) the DNA sequence (i) has the length of at least 23 nucleobases and comprises SEQ ID NO:1, or a variant thereof; wherein said variant has at least 80% sequence identity with SEQ ID NO:1;
b) the promoter is an isolated DNA sequence that comprises a single binding site for cyclic AMP receptor protein (CRP) centred at position around −41 from transcription start.

In some embodiments the CRP binding site comprises SEQ ID NO: 51 or SEQ ID NO: 52, or variants thereof. In some embodiments the promoter DNA sequence consists of or comprise SEQ ID NO: 21 or SEQ ID NO: 22, or a variant or fragment thereof. In some embodiments the construct further comprises a DNA sequence (ii), wherein said DNA sequence (ii) is operably linked downstream the DNA sequence (i). In one embodiment, the DNA sequence (ii) is a non-coding DNA sequence. Preferably, the non-coding DNA sequence comprises a ribosomal binding site (RBS). The RBS binding site of the non-coding DNA sequence may different embodiments comprise a DNA sequence selected from any of SEQ ID NOs: 3-20. The construct comprising an RNA sequence, may further comprise a coding DNA sequence which is operably linked to the non-coding DNA sequence (ii). Preferably, the coding DNA sequence of such construct encodes a polypeptide. The polypeptide may be an enzyme, transport protein, antigen, regulatory protein.

In some embodiments of the nucleic acid construct of above, the DNA sequence (ii) is a coding DNA sequence. The coding DNA sequence (ii) preferably comprise a DNA sequence encoding a small non-coding RNA molecule, such as a regulatory microRNA (miRNA) or small interfering RNA (siRNA) molecule.

In another embodiment, a nucleic acid construct comprises a contiguous synthetic nucleic acid that comprising two DNA sequences (i) and (ii), wherein the DNA sequence (ii) is operably linked downstream the DNA sequence (i), and
wherein
   a) the DNA sequence (i) has the length of at least 23 nucleobases and comprises SEQ ID NO:1, or a variant thereof, wherein the variant has at least 80% sequence identity with SEQ ID NO:1;
   b) the DNA sequence (ii) does not comprise any of the sequences of SEQ ID NOs: 3-18;

This nucleic acid construct may further comprises a promoter that is operably linked to the DNA sequence (i). The promoter of such nucleic acid construct comprises an isolated DNA sequence that may, in one preferred embodiment, comprises a single binding site cyclic AMP receptor protein (CRP) centred at position around −41 upstream the transcription start point. In some preferred embodiments, the CRP binding site comprises SEQ ID NO: 51 or SEQ ID NO: 52, or variants thereof. The promoter DNA sequence may have a sequence of SEQ ID NO: 21 or SEQ ID NO: 22, or a variant or fragment thereof, or comprises said sequences. The nucleic acid construct may further comprise a coding DNA sequence that encodes a functional polypeptide, such as an enzyme, transport protein, antigen, regulatory protein, or a small non-coding RNA molecule, such as a regulatory microRNA (miRNA) or small interfering RNA (siRNA) molecule. The later construct preferably comprises a DNA sequence (ii) that comprises a ribosomal binding site. In some preferred embodiments, the ribosomal binding site may comprise SEQ ID NO:19 or 20.

In one embodiment, the invention relates to a vector comprising a nucleic acid sequence od SEQ ID NO: 1, or a variant thereof, wherein the variant has at least 80% sequence identity with SEQ ID NO:1. In another embodiment, the invention relates to a vector comprising a nucleic acid construct as any of the described above.

In one embodiment, the invention relates to an expression cassette comprising a nucleic acid sequence of SEQ ID NO: 1, or a variant thereof, wherein the variant has at least 80% sequence identity with SEQ ID NO:1. In another embodiment, the invention relates to an expression cassette that comprises a construct as any of the described above.

In one embodiment, the invention relates to a recombinant cell that in different embodiments may comprise a nucleic acid construct, vector, expression cassette as any of the described above. Preferably, the cell is a bacterial cell.

In one embodiment, the invention relates to an expression system that may in different embodiments comprise a nucleic acid sequence, a construct and/or a recombinant cell and any of the described above.

In one embodiment, the invention relates to a method of recombinant production of a biological molecule, preferably, a protein, such as an enzyme, transporter protein, a regulatory protein, structural protein, or a small non-coding RNA molecule, such as a regulatory microRNA (miRNA) or small interfering RNA (siRNA) molecule, or an oligosaccharide, such as a human milk oligosaccharide, comprising
(a) providing a nucleic acid or a construct as described above,
(b) providing a recombinant cell or an expression system as described above;
(c) producing the biological molecule in the cell or expression system of (b).

### EXAMPLES

Materials and Methods

Unless otherwise noted, standard techniques, vectors, control sequence elements, and other expression system elements known in the field of molecular biology are used for nucleic acid manipulation, transformation, and expression. Such standard techniques, vectors, and elements can be found, for example, in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY)

Strains and Plasmids

The bacterial strain used, MDO, was constructed from Escherichia coli K12 DH1. The E. coli K12 DH1 genotype is: F⁻, λ⁻, gyrA96, recA1, relA1, endA1, thi-1, hsdR17, supE44. In addition to the E. coli K12 DH1 genotype MDO has the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Strains utilized in the present Examples are described in Table 5. Donor and helper plasmids used for the construction of these strains are enlisted in Table 6. Primers used for construction of plasmids are listed in Table 7.

TABLE 5

| | |
|---|---|
| Strain IDs | Genomic Description |

Background Strains

| | |
|---|---|
| DH1 | F⁻ λ⁻ endA1 recA1 relA1 gyrA96 thi-1 glnV44 hsdR17 |
| | (r_{K}⁻m_{K}⁻) |
| MDO | E coli DH1 ΔlacZ, ΔlacA, ΔnanKETA, ΔmelA, ΔwcaJ, |
| | ΔmdoH |

Strains expressing reporter gene IacZ

| | |
|---|---|
| MAP292 | MDO Plac-futC-T1 |
| MAP1021 | MDO galK::PmglB_16UTR-IacZ-T1 |
| MAP1730 | MDO galK::PmglB 70UTR-IacZ-T1 |
| MAP1739 | MDO galK::PmglB_70UTR_SD4-IacZ-T1 |
| MAP1740 | MDO galK::PmglB_70UTR_SD5-IacZ-T1 |
| MAP1741 | MDO galK::PmglB_70UTR_SD7-IacZ-T1 |
| MAP1742 | MDO galK::PmglB_70UTR_SD8-IacZ-T1 |
| MAP1743 | MDO galK::PmglB_70UTR_SD9-IacZ-T1 |
| MAP1918 | MDO galK::PmglB_org-IacZ-T1 |
| MAP1919 | MDO galK::PgatY_org-IacZ-T1 |
| MAP1920 | MDO galK::PmglB_54UTR-IacZ-T1 |
| MAP1921 | MDO galK::PgatY_54UTR-IacZ-T1 |
| MAP1994 | MDO PmglB_70UTR_SD4-futC-T1 |

TABLE 6

| Plasmid ID | Description |
|---|---|
| pACBSR | Para-I-Scel-λ Red, p15A ori, cam* |
| pUC57 | pMB1, bla |
| pUC57::gal | pUC57::galTK’/T1-galKM’ |
| pMAP409 | pUC57::galTK’-PmglB_16UTR-IacZ-T1-galKM’ |
| pMAP1030 | pUC57::galTK’-PmglB_70UTR-IacZ-T1-galKM’ |
| pMAP1069 | pUC57::galTK’-PmglB_70UTR_SD4-IacZ-T1-galKM’ |
| pMAP1070 | pUC57::galTK’-PmglB_70UTR_SD5-IacZ-T1-galKM’ |
| pMAP1071 | pUC57::galTK’-PmglB_70UTR_SD7-IacZ-T1-galKM’ |
| pMAP1072 | pUC57::galTK’-PmglB_70UTR_SD8-IacZ-T1-galKM’ |
| pMAP1073 | pUC57::galTK’-PmglB_70UTR_SD9-IacZ-T1-galKM’ |
| pMAP1226 | pUC57-galTK’-PmglB_org-IacZ-T1-galKM’ |
| pMAP1227 | pUC57-galTK’-PgatY_org-IacZ-T1-galKM’ |
| pMAP1229 | pUC57-galTK’-PmglB_54UTR-IacZ-T1-galKM’ |
| pMAP1230 | pUC57-galTK’-PgatY_54UTR-IacZ-T1-galKM’ |

Media

The Luria Broth (LB) medium was made using LB Broth Powder, Millers (Fisher Scientific) and LB agar plates were made using LB Agar Powder, Millers (Fisher Scientific). Screening of strains on LB plates containing 5-Bromo-4-chloro-3-indolyl-β-D-galactopyranoside (X-gal) was done using an X-gal concentration of 40 μg/ml. When appropriated ampicillin (100 μg/ml) and/or chloramphenicol (20 μg/ml) was added.

Basal Minimal medium had the following composition: NaOH (1 g/L), KOH (2.5 g/L), KH₂PO₄ (7 g/L), NH₄H₂PO₄ (7 g/L), Citric acid (0.5 g/l), Trace mineral solution (5 ml/L). The trace mineral stock solution contained: ZnSO₄.7H₂O 0.82 g/L, Citric acid 20 g/L, MnSO₄.H₂O 0.98 g/L, FeSO₄.7H₂O 3.925 g/L, CuSO₄.5H₂O 0.2 g/L. The pH of the Basal Minimal Medium was adjusted to 7.0 with 5 N NaOH and autoclaved. Before inoculation the Basal Minimal medium was supplied with 1 mM MgSO₄, 4 μg/ml thiamine, 0.5% of a given carbon source (glucose or glycerol (Carbosynth)), and when appropriated ampicillin (100 μg/ml) and/or chloramphenicol (20 μg/ml) was added. Thiamine and antibiotics were sterilized by filtration. All percentage concentrations for glycerol are expressed as v/v and those for glucose as w/v. M9 plates containing 2-deoxy-galactose had the following composition: 15 g/L agar (Fisher Scientific), 2.26 g/L 5×M9 Minimal Salt (Sigma-Aldrich), 2 mM MgSO4, 4 μg/ml thiamine, 0.2% glycerol, and 0.2% 2-deoxy-D-galactose (Carbosynth).

MacConkey indicator plates containing galactose had the following composition: 40 g/L MacConkey agar Base (BD Difco™). After autoclaving and cooling to 50° C., D-galactose (Carbosynth) was added to a final concentration of 1%.

Cultivation

Unless otherwise noted, E. coli strains were propagated in Luria-Bertani (LB) medium containing 0.2% glucose at 37° C. with agitation.

Cultures harvested for β-galactosidase assays were made in the following way: A single colony from an LB-plate was pre-cultured in 1 ml Basal Minimum media containing glucose (0.5%) in a 10 ml 24 Deep well plate (Axygen). The plate was sealed before culturing with a Hydrophobic Gas Permeable Adhesive Seal (Axygen) and incubated for 24 hours at 34° C. with shaking at 700 rpm in an orbital shaker (Edmund Buhler GmbH). Cell density of the culture was monitored at 600 nm using an S-20 spectrophotometer (Boeco, Germany). 20 μl of the overnight culture was used for inoculation in 2 ml LB or Basal Minimum media containing glucose or another carbon source (0.5%) in a 24 Deep well plate. The Deep well plates were covered with sealing foil and incubated for 24 hours at 28° C. with orbital shaking at 700 rpm. After incubation, OD600 was measured and 0.5 ml cell culture was harvested by centrifugation for preforming β-galactosidase assay.

Chemical Competent Cells and Transformations

E. coli was inoculated from LB plates in 5 ml LB containing 0.2% glucose at 37° C. with shaking until OD600 ˜0.4. 2 ml culture was harvested by centrifugation for 25 seconds at 13.000 g. The supernatant was removed, and the cell pellet resuspended in 600 ul cold TB solutions (10 mM PIPES, 15 mM CaCl₂, 250 mM KCl). The cells were incubated on ice for 20 minutes followed by pelleting for 15 seconds at 13.000 g. The supernatant was removed, and the cell pellet resuspended in 100 μl cold TB solution. Transformation of plasmids were done using 100 μl competent cells and 1-10 ng plasmid DNA. Cells and DNA were incubated on ice for 20 minutes before heat shocking at 42° C. for 45 seconds. After 2 min incubation on ice 400 μl SOC (20 g/L tryptone, 5 g/L Yeast extract, 0.5 g/L NaCl, 0.186 g/L KCl, 10 mM MgCl₂, 10 mM MgSO₄ and 20 mM glucose) was added and the cell culture was incubated at 37° C. with shaking for 1 hour before plating on selective plates.

Plasmid ligations were transformed into TOP10 chemical competent cells at conditions recommended by the supplier (ThermoFisher Scientific).

DNA Techniques

Plasmid DNA from E. coli was isolated using the QIAprep Spin Miniprep kit (Qiagen). Chromosomal DNA from E. coli was isolated using the QIAmp DNA Mini Kit (Qiagen). PCR products were purified using the QIAquick PCR Purification Kit (Qiagen). DreamTaq PCR Master Mix (Thermofisher), Phusion U hot start PCR master mix (Thermofisher), USER Enzym (New England Biolab) were used as recommended by the supplier. Primers were supplied by Eurofins Genomics, Germany. PCR fragments and plasmids were sequenced by Eurofins Genomics.

Colony PCR was done using DreamTaq PCR Master Mix, at conditions recommended by the supplier (Thermofisher) in a T100™ Thermal Cycler (Bio-Rad). For instance, during the construction of strains expressing a reporter or recombinant gene from the galK locus, primers O48 and O49 were used in a colony PCR reaction aiming to confirm the validity of the intended modification.

TABLE 7

| Name | Oligonucleotide Sequence 5′-3′ | Description | ID |
|---|---|---|---|
| O40 | ATTAACCCUCCAGGCATCAAATAAAACGAAAGGC | Backbone.for | 32 |
| O48 | CCCAGCGAGACCTGACCGCAGAAC | gal.for | 33 |
| O49 | CCCCAGTCCATCAGCGTGACTACC | gal.rev | 34 |
| O77 | AAACAGCUATGACCATGATTACGGATTC | lacZ.for | 35 |
| O78 | AGGGTTAAUTGCGCGTTATTTTTGACACCAGACCAA | lacZ.rev | 36 |
| | CTGG | | |
| O79 | ATTTGCGCAUCACCAATCAAATTCACGCGGCC | Backbone.rev | 37 |
| O362 | ATGCGCAAAUCGGCAACCTATGCCTGATGCGACGC | PgatY.for | 38 |
| O364 | ATGCGCAAAUTGCGTCGCCATTCTGTCGCAACACGC | PmglB.for | 39 |
| | C | | |
| O365 | AGCTGTTUCCTCCTTGCTTATGCAGGGTAGTGCTTG | PmglB_16UTR.rev | 40 |
| | AGATAAATG | | |
| O459 | AGCTGTTUcctagTTGGTTAATGTTTGTTGTATGCG | PmglB_70UTR_SD4.rev | 41 |
| O460 | AGCTGTTUctcggTTGGTTAATGTTTGTTGTATGCG | PmglB_70UTR_SD5.rev | 42 |
| O462 | AGCTGTTUtgctcTTGGTTAATGTTTGTTGTATGCG | PmglB_70UTR_SD7.rev | 43 |
| O463 | AGCTGTTUttctcTTGGTTAATGTTTGTTGTATGCG | PmglB_70UTR_SD8.rev | 44 |
| O464 | AGCTGTTUttcctTTGGTTAATGTTTGTTGTATGCG | PmglB_70UTR_SD9.rev | 45 |
| O990 | AGCTGTTUCCTCCTTGGTTAATGTTTGTTGTATGCG | PmglB_70UTR.rev | 46 |
| | TGAAAGTCACGGACCTCCACGATGCTTGTAGGCACG | | |
| | GTGCAATCATAGCTATCACATTG | | |
| OL-090 | ATGGTCAUGGTATCTCCGGTTTTTCTTATGCAGGG | PmglB_org.rev | 47 |
| OL-091 | ATGGTCAUTTTCATATCCTGTCGTTTGTTTTCG | PgatY_org.rev | 48 |
| OL-092 | ATGGTCAUGGTATCTCCGGTTTTTGTTAATGTTTGT | PmglB_54UTR.rev | 49 |
| | TGTATGCGTGAAAGTCACGGACCTCCACGATGCTTG | | |
| | TAGGCACGGTGCAATCATAGCTATCACATTG | | |
| OL-093 | ATGGTCAUTTTCATATCCTGTCGTGTTAATGTTTGT | PgatY_54UTR.rev | 50 |
| | TGTATGCGTGAAAGTCACGGACCTCCACGATGCTTG | | |
| | TAGGCACAAAATATAATGAAATTATTTG | | |

Construction of Plasmids

A plasmid containing two I-Sce/endonuclease sites, separated by two DNA fragments of the gal operon (required for homologous recombination in galK), and a T1 transcriptional terminator sequence (pUC57::gal) was synthesized (GeneScript). The DNA sequences used for homologous recombination in the gal operon covered base pairs 3.628.621-3.628.720 and 3.627.572-3.627.671 in sequence Escherichia coli K12 MG155 complete genome GenBank: ID: CP014225.1. Insertion by homologous recombination would result in a deletion of 949 base pairs of galK and a galK-phenotype.

Standard techniques well-known in the field of molecular biology were used for designing primers and amplification of specific DNA sequences of the Escherichia coli K-12 DH1 chromosomal DNA. Such standard techniques, vectors, and elements can be found, for example, in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.)

A 3.5 kbp plasmid backbone containing pUC57-scel-galTK-T1-galKM-scel, was amplified using primers O40 and O79 and a 3.3 kbp DNA fragment containing lacZ was amplified from chromosomal DNA isolated from E. coli K-12 DH1 using primers and O78.

Chromosomal DNA obtained from E. coli DH1 or constructed plasmids were used to amplify DNA fragments containing promoter elements. DNA fragments containing the promoter PgatY_org (SEQ ID NO:21) was amplified using O362 and OL-091; PgatY_54UTR (SEQ ID NO:24) using O362 and OL-093 and pMAP1227 as DNA template; PmglB_org (SEQ ID NO: 22) using O364 and OL-090; PmglB 16UTR (SEQ ID NO: 25) using O364 and O365; PmglB_54UTR (SEQ ID NO: 24) using O364 and OL-092 and pMAP1226 as DNA template; PmglB_70UTR (SEQ ID NO: 26) using O364 and O990 and pMAP409 as DNA template; PmglB_70UTR_SD4 (SEQ ID NO: 27) using O364 and O459 and pMAP1030 as DNA template, PmglB_70UTR_SD5 (SEQ ID NO: 28) using O364 and O460 and pMAP1030 as DNA template; PmglB_70UTR_SD7 (SEQ ID NO: 29) using O364 and O462 and pMAP1030 as DNA template; PmglB_70UTR_SD8 (SEQ ID NO: 30) using O364 and O463 and pMAP1030 as DNA template; PmglB_70UTR_SD9 (SEQ ID NO: 31) using O364 and O464 and pMAP1030 as DNA template.

All PCR fragments were purified, and plasmid backbone, promoter elements, and lacZ were cloned, transformed into TOP10 cells and selected on LB plates containing 100 μl/ml ampicillin and 0.2% glucose. The constructed plasmids (see Table 6) were purified. The promoter sequence and the 5′end of the lacZ gene was verified by DNA sequencing (MWG Eurofins Genomics).

All plasmid backbones constructed contained two specific DNA fragments homologous to Escherichia coli K-12 DH1 used for homologous recombination. In this way, a genetic cassette comprising any promoter construct of interest, lacZ, and the T1 transcriptional terminator was inserted specifically in the Escherichia coli genome. Construction of plasmids used for recombineering was done using standard cloning techniques. The DNA sequence of the expression elements is shown in Table 8.

TABLE 8

| SEQ name | Nucleotide Sequence 5′-3′ | ID |
|---|---|---|
| 23UTR-glpF | CGTGGAGGTCCGTGACTTTCACG | 1 |
| 54UTR-glpF | TGCCTACAAGCATCGTGGAGGTCCGTGACTTTCACGCATACAACAAACATTA | 2 |
| | AC | |
| PgatY_org | CGGCAACCTATGCCTGATGCGACGCTGAAGCGTCTTATCATGCCTACATAGC | 21 |
| | ACTGCCACGTATGTTTACACCGCATCCGGCATAAAAACACGCGCACTTTGCT | |
| | ACGGCTTCCCTATCGGGAGGCCGTTTTTTTGCCTTTCACTCCTCGAATAATT | |
| | TTCATATTGTCGTTTTTGTGATCGTTATCTCGATATTTAAAAACAAATAATT | |
| | TCATTATATTTTGAAATCGAAAACAAACGACAGGATATGAAA | |
| PmglB_org | TGCGTCGCCATTCTGTCGCAACACGCCAGAATGCGGCGGCGATCACTAACTC | 22 |
| | AACAAATCAGGCGATGTAACCGCTTTCAATCTGTGAGTGATTTCACAGTATC | |
| | TTAACAATGTGATAGCTATGATTGCACCGTTTTAACGTTGTAACCCGTATGT | |
| | AACAGTGAATAATCACTTTTGCCGAGGTAACAGCGTCATAACAACAATTAAA | |
| | GCCGTTTTCTGGAGCGTTACCGGGCATGGAAGAACGAATTTTAAAAAGTGAG | |
| | CTTCGGCGTTCAGTAACACTTCATTAACTCTACTGCCCCGCCGAGCATTTAT | |
| | CTCAAGCACTACCCTGCATAAGAAAAACCGGAGATACC | |
| PgatY_54UTR | CGGCAACCTATGCCTGATGCGACGCTGAAGCGTCTTATCATGCCTACATAGC | 23 |
| | ACTGCCACGTATGTTTACACCGCATCCGGCATAAAAACACGCGCACTTTGCT | |
| | ACGGCTTCCCTATCGGGAGGCCGTTTTTTTGCCTTTCACTCCTCGAATAATT | |
| | TTCATATTGTCGTTTTTGTGATCGTTATCTCGATATTTAAAAACAAATAATT | |
| | TCATTATATTTTGTGCCTACAAGCATCGTGGAGGTCCGTGACTTTCACGCAT | |
| | ACAACAAACATTAACACGACAGGATATGAAA | |
| PmglB_54UTR | TGCGTCGCCATTCTGTCGCAACACGCCAGAATGCGGCGGCGATCACTAACTC | 24 |
| | AACAAATCAGGCGATGTAACCGCTTTCAATCTGTGAGTGATTTCACAGTATC | |
| | TTAACAATGTGATAGCTATGATTGCACCGTGCCTACAAGCATCGTGGAGGTC | |
| | CGTGACTTTCACGCATACAACAAACATTAACAAAAACCGGAGATACC | |
| PmglB_16UTR | TGCGTCGCCATTCTGTCGCAACACGCCAGAATGCGGCGGCGATCACTAACTC | 25 |
| | AACAAATCAGGCGATGTAACCGCTTTCAATCTGTGAGTGATTTCACAGTATC | |
| | TTAACAATGTGATAGCTATGATTGCACCGTTTTAACGTTGTAACCCGTATGT | |
| | AACAGTGAATAATCACTTTTGCCGAGGTAACAGCGTCATAACAACAATTAAA | |
| | GCCGTTTTCTGGAGCGTTACCGGGCATGGAAGAACGAATTTTAAAAAGTGAG | |
| | CTTCGGCGTTCAGTAACACTTCATTAACTCTACTGCCCCGCCGAGCATTTAT | |
| | CTCAAGCACTACCCTGCATAAGCAAGGAGGAAACAGCT | |
| PmglB_70UTR | TGCGTCGCCATTCTGTCGCAACACGCCAGAATGCGGCGGCGATCACTAACTC | 26 |
| | AACAAATCAGGCGATGTAACCGCTTTCAATCTGTGAGTGATTTCACAGTATC | |
| | TTAACAATGTGATAGCTATGATTGCACCGTGCCTACAAGCATCGTGGAGGTC | |
| | CGTGACTTTCACGCATACAACAAACATTAACCAAGGAGGAAACAGCT | |
| PmglB_70UTR_SD4 | TGCGTCGCCATTCTGTCGCAACACGCCAGAATGCGGCGGCGATCACTAACTC | 27 |
| | AACAAATCAGGCGATGTAACCGCTTTCAATCTGTGAGTGATTTCACAGTATC | |
| | TTAACAATGTGATAGCTATGATTGCACCGTGCCTACAAGCATCGTGGAGGTC | |
| | CGTGACTTTCACGCATACAACAAACATTAACCAACTAGGAAACAGCT | |
| PmglB_70UTR_SD5 | TGCGTCGCCATTCTGTCGCAACACGCCAGAATGCGGCGGCGATCACTAACTC | 28 |
| | AACAAATCAGGCGATGTAACCGCTTTCAATCTGTGAGTGATTTCACAGTATC | |
| | TTAACAATGTGATAGCTATGATTGCACCGTGCCTACAAGCATCGTGGAGGTC | |
| | CGTGACTTTCACGCATACAACAAACATTAACCAACCGAGAAACAGCT | |
| PmglB_70UTR_SD7 | TGCGTCGCCATTCTGTCGCAACACGCCAGAATGCGGCGGCGATCACTAACTC | 29 |
| | AACAAATCAGGCGATGTAACCGCTTTCAATCTGTGAGTGATTTCACAGTATC | |
| | TTAACAATGTGATAGCTATGATTGCACCGTGCCTACAAGCATCGTGGAGGTC | |
| | CGTGACTTTCACGCATACAACAAACATTAACCAAGAGCAAAACAGCT | |
| PmglB_70UTR_SD8 | TGCGTCGCCATTCTGTCGCAACACGCCAGAATGCGGCGGCGATCACTAACTC | 30 |
| | AACAAATCAGGCGATGTAACCGCTTTCAATCTGTGAGTGATTTCACAGTATC | |
| | TTAACAATGTGATAGCTATGATTGCACCGTGCCTACAAGCATCGTGGAGGTC | |
| | CGTGACTTTCACGCATACAACAAACATTAACCAAGAGAAAAACAGCT | |
| PmglB_70UTR_SD9 | TGCGTCGCCATTCTGTCGCAACACGCCAGAATGCGGCGGCGATCACTAACTC | 31 |
| | AACAAATCAGGCGATGTAACCGCTTTCAATCTGTGAGTGATTTCACAGTATC | |
| | TTAACAATGTGATAGCTATGATTGCACCGTGCCTACAAGCATCGTGGAGGTC | |
| | CGTGACTTTCACGCATACAACAAACATTAACCAAAGGAAAAACAGCT | |

Construction of Strains

Insertion of promoter expression elements fused to a reporter gene or a recombinant gene was performed by Gene Gorging essentially as described by Herring et al (Herring, C. D., Glasner, J. D. and Blattner, F. R. (2003). Gene (311). 153-163). Briefly, the donor plasmid and the helper plasmid were co-transformed into MDO and selected on LB plates containing 0.2% glucose, ampicillin (100 μg/ml) or kanamycin (50 mg/mL) and chloramphenicol (20 μg/ml). A single colony was inoculated in 1 ml LB containing chloramphenicol (20 μg/ml) and 10 μl of 20% L-arabinose and incubated at 37° C. with shaking for 7-8 hours. Cells were then plated on M9-DOG plates and incubated at 37° C. for 48 hours. Single colonies formed on MM-DOG plates were re-streaked on LB plates containing 0.2% glucose and incubated for 24 hours at 37° C.

For insertion at the galK locus, colonies that appeared white on MacConkey-galactose agar plates, and were sensitive for both ampicillin and chloramphenicol were expected to have lost the donor and the helper plasmid, and contain an insertion in the galK loci. Insertions in the galK site was identified by colony PCR using primers 048 and 049 located outside the galK loci. Chromosomal DNA was purified, the galK locus was amplified using primers 048 and 049 and the inserted DNA was verified by sequencing (Eurofins Genomics, Germany).

Strains MAP1021, MAP1730, MAP1739, MAP1740, MAP1741, MAP1742, MAP1743, MAP1918, MAP1919, MAP1920, and MAP1921 were constructed using donor plasmids pMAP409, pMAP1030, pMAP1069, pMAP1070, pMAP1071, pMAP1072, pMAP1073, pMAP1226, pMAP1227, pMAP1229, and pMAP1230, respectively.

Enzyme Assay: lacZ

The β-Galactosidase activity was assayed as described previously (see e.g. Miller J. H. Experiments in molecular genetics, Cold spring Harbor Laboratory Press, N Y, 1972). Briefly the cells were diluted in Z-buffer and permeabilized with sodium dodecyl sulfate (0.1%) and chloroform. Assays were performed at 30° C. Samples were preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol was subsequently determined as the change in optical density at 420 nm. The specific activities are reported in Miller Units [A420/(min*ml*A600)]. The activities are average values from at least two independent experiments.

### Example 1—Modulating Gene Expression by Replacing Part of the 5′UTR with Synthetic DNA Comprising 54UTR-glpF in the Expression Elements PgatY_Org, and PmglB_Org, Respectively

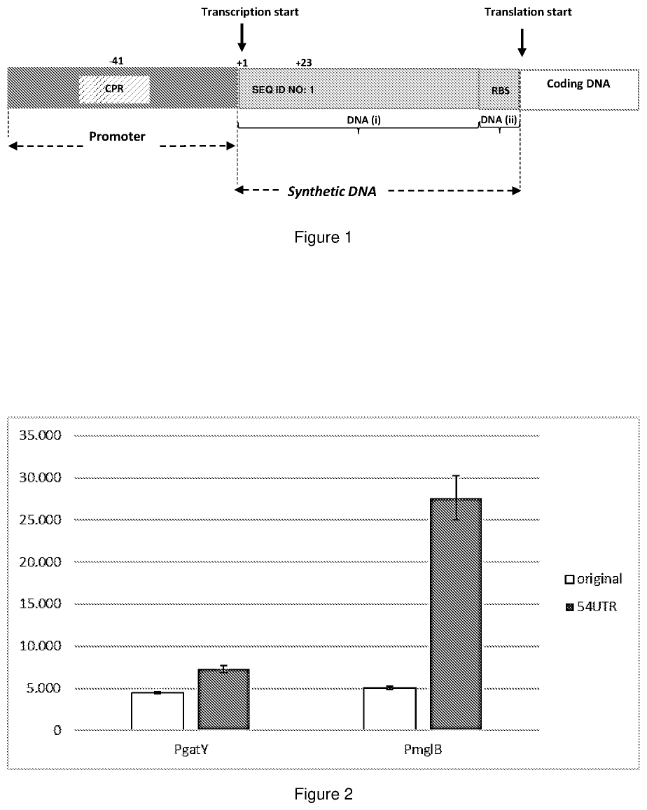

A promoter-probe plasmid containing a promoter-less lacZ gene was used to clone four DNA fragments comprising various promoter elements. The expression levels of lacZ was determined after fusion of a promoter element to lacZ followed by integration of the Promoter-lacZ element in a single copy into the chromosomal DNA. The AlacZM15 deletion in the lacZ gene in E. coli MDO makes it unable to produce an active β-galactosidase enzyme and was therefore used as strain background in the screen. Two recombinant nucleic acid sequences comprising the genomic promoter sequences originating from the operons gatYZABCDR, and mglBAC, were fused to promoter-less lacZ reporter gene and inserted into the chromosomal DNA in a single copy. The expression level of the cloned fragment was measured (FIG. 1, white bars). The 5′UTR regions in the expression elements PgatY_org (SEQ ID NO: 21), and PmglB_org (SEQ ID NO: 22) were modified by replacing the 5′UTR between the transcriptional start site and 16 bp upstream of the translation start site with the 54-nucleotide long fragment 54UTR-glpF (SEQ ID NO: 2), i.e. resulting in gatY-54UTR-glpF(SEQ ID NO: 23), and mglB-54UTR-glpF (SEQ ID NO: 24). Surprisingly, replacement of the 5′UTR regions of PgatY_org, and PmglB_org with 54UTR-glpF increased expression (FIG. 2, grey bars) suggesting that glpF-54UTR (SEQ ID NO: 2) may be used as a tool for modulating gene expression from a variety, if not any, promoter and from any construct.

### Example 2—Use of a Synthetic PmglB Expression Element for Modulating Expression of Recombinant Nucleic Acid Sequences

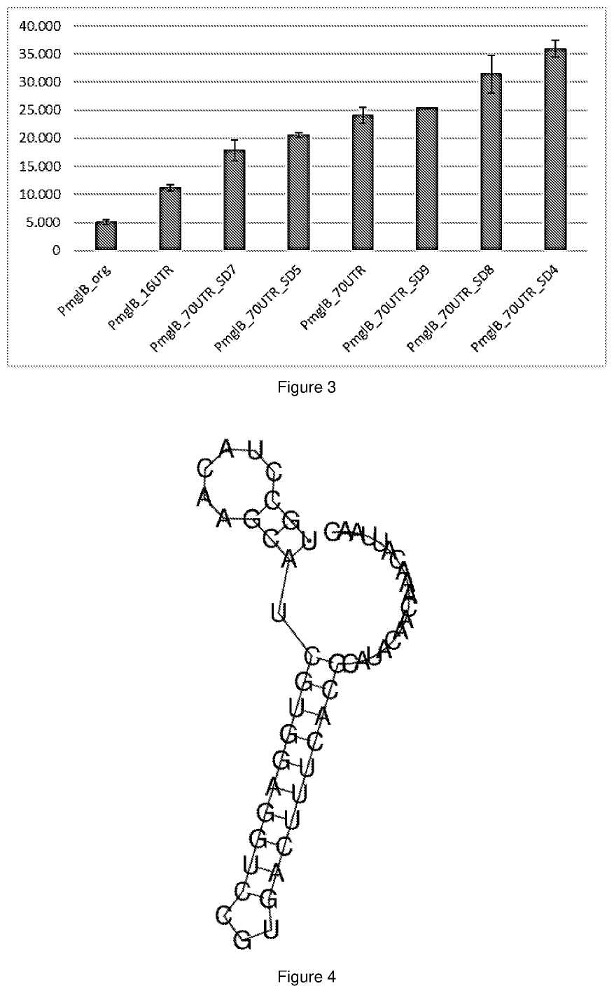

We have previously demonstrated the effect of modifications of 16UTR/Rec UTR (SEQ ID NO: 3) sequence on gene expression from PglpF_70UTR and PglpT_70UTR constructs comprising this sequence (PCT/IB2018/060355). Here we confirm that the variants of 16UTR combined with 54UTR-glpF described in PCT/IB2018/060355 have a similar effect of expression of the lacZ gene from constructs comprising PmglB. Changing the 16 nucleotide DNA fragment of the mglB-5′URT located directly upstream of the translation start site of mglB with a synthetic DNA fragment (16UTR, SEQ ID NO: 3), increase expression 2-fold. Replacing the entire mglB 5′UTR region located between the transcriptional start site and the translational start codon with the glpF-70UTR sequence, resulting in PmglB_70UTR (SEQ ID NO: 25), increase expression level almost 5-fold compared to the original promoter element, PmglB_org. Furthermore, minor variations in the nucleotide sequence of 16UTR that comprises the ribosomal binding site (RBS or Shine Dalgarno sequence) had a significant effect on the expression level from PmglB_70UTR, resulting in an expression library where the expression levels varied up to 7-fold (FIG. 3).

### Example 3

A secondary structure of the 5′RNA transcript of SEQ ID NO:2 was analysed using the RNAfold WebServer (http://rna.tbi.univie.ac.at/cgi-bin/RNAWebSuite/RNAfold.cgi) and RNAstructure Predict (http://rna.urmc.rochester.edu/RNAstructure.html). It was found that a twenty-three-nucleotide fragment of this sequence (SEQ ID NO: 1) forms a pin structure as shown in FIG. 4. Without been bound to a theory, we suggest herein that a transcript of SEQ ID NO: 1 stabilizes an RNA molecule comprising thereof.

### Example 4

In a strain background, where the colonic acid genes gmd, wcaJ (fcl), cpsB (manC) and cpsG (manB) are overexpressed from Plac, a single copy of futC was expressed either from Plac promoter (strain MAP292) or from PmglB_70UTR_SD4 promoter (SEQ ID NO: 27) (strain MAP1994). Analysis of the 2′-FL production under feed-batch conditions showed that 2′-FL titres increased 3-7-fold when futC is expressed form PmglB_70UTR_SD4 (data not shown).

1. (canceled)

2. A nucleic acid construct comprising a promoter DNA sequence that is operably linked to a contiguous synthetic DNA sequence (i),
wherein
a) the DNA sequence (i) has the length of at least 23 nucleobases and comprises SEQ ID NO:1, or a variant thereof; wherein said variant has at least 80% sequence identity with SEQ ID NO:1; and
b) the promoter is an isolated DNA sequence that comprises a single binding site cyclic AMP receptor protein (CRP) centred at position around −41, upstream the transcription start point.

3. The nucleic acid construct of claim 2, wherein the CRP binding site comprising SEQ ID NO: 51, SEQ ID NO: 52, or a variant thereof.

4. The nucleic acid construct of claim 2, wherein the promoter DNA sequence comprises SEQ ID NO: 21, SEQ ID NO: 22, or a variant or fragment thereof.

5. The nucleic acid construct of claim 2, wherein the construct further comprises a DNA sequence (ii), wherein said DNA sequence (ii) is operably linked to the DNA sequence (i).

6. The nucleic acid construct of claim 5, wherein the DNA sequence (ii) comprises a non-coding DNA sequence comprising a ribosomal binding site.

7. (canceled)

8. The nucleic acid construct of claim 7, wherein the non-coding DNA sequence comprises a sequence selected from the group consisting of SEQ ID NOs: 3, 4, 5, 6, 7, 8, 9, 10, 11, 12, 13, 14, 15, 16, 17, 18, 19 and 20.

9. The nucleic acid construct of claim 7, wherein the construct further comprises a coding DNA sequence which is operably linked to the DNA sequence (ii), and wherein the coding DNA sequence encodes a polypeptide.

10. (canceled)

11. The nucleic acid construct of claim 9, wherein the polypeptide is an enzyme, transport protein, antigen or regulatory protein.

12. The nucleic acid construct of claim 5, wherein the DNA sequence (ii) comprises a coding DNA sequence that encodes a small non-coding RNA molecule.

13. The nucleic acid construct of claim 12, wherein the small non-coding RNA molecule is a regulatory microRNA (miRNA) or small interfering RNA (siRNA) molecule.

14. A nucleic acid construct comprising a contiguous synthetic nucleic acid comprising two DNA sequences (i) and (ii), wherein the DNA sequence (i) is operably linked the DNA sequence (ii), and wherein
(a) the DNA sequence (i) has the length of at least 23 nucleobases and comprises SEQ ID NO:1, or a variant thereof, wherein said variant has at least 80% sequence identity with SEQ ID NO:1; and
(b) the DNA sequence (ii) does not comprise any of the sequences of SEQ ID NOs: 3 to 18.

15. The nucleic acid construct of claim 14, wherein the construct further comprises a promoter that is operably linked to the DNA sequence (i).

16. The nucleic acid construct of claim 15, wherein the promoter is an isolated DNA sequence that comprises a single binding site for cyclic AMP receptor protein (CRP) centred at position around −41, upstream the transcription start point.

17. The nucleic acid construct of claim 16, wherein the CRP binding site comprising SEQ ID NO: 51 or SEQ ID NO: 52, or a variant thereof.

18. The nucleic acid construct of claim 16, wherein the promoter DNA sequence comprises SEQ ID NO: 21 or SEQ ID NO: 22, or a variant or fragment thereof.

19. The nucleic acid construct of claim 15, wherein the nucleic acid construct further comprises a coding DNA sequence that is operably linked to DNA sequence (ii), wherein said coding DNA sequence encodes a small non-coding RNA molecule.

20. The nucleic acid construct of claim 15, wherein the construct comprises a coding DNA sequence that that is operably linked to DNA sequence (ii), wherein said coding DNA encodes a functional polypeptide, and wherein the DNA sequence (ii) comprises a ribosomal binding site.

21.-23. (canceled)

24. A recombinant cell comprising a vector expression cassette comprising a nucleic acid construct claim 2.

25.-26. (canceled)

27. A method of recombinant production of one or more biological molecules, comprising
(a) providing a recombinant cell of claim 24;
(b) producing the one or more biological molecules in the cell of (a).

28. The method of claim 27, wherein the one or more biological molecules is
a protein,
a small non-coding RNA molecule, or
an oligosaccharide.
